(19) United States
(12) Patent Application Publication
(10) Pub. No.: US 2004/0102382 A1
(43) Pub. Date: May 27, 2004

(54) Targeting peptides

(75) Inventors: Klaus Schughart, Kenzingen (DE); Ulla Rasmussen, Achenheim (FR); Valerie Schreiber, Graffenstaden (FR)

Correspondence Address: BURNS DOANE SWECKER & MATHIS L L P, POST OFFICE BOX 1404, ALEXANDRIA, VA, 22313-1404, US

(73) Assignee: TRANSGENE, S.A., STRASBOURG CEDEX (FR)

(21) Appl. No.: 10/346,058

(22) Filed: Jan. 17, 2003

Related U.S. Application Data
(63) Continuation-in-part of application No. 10/182,573, filed on Nov. 27, 2002.

(51) Int. Cl. A61K 38/08; A61K 38/10; C07K 7/08

(52) U.S. Cl. 514/14; 514/15; 514/16; 530/326; 530/327; 530/328

(57) ABSTRACT

The present invention relates to targeting peptides and more specifically peptides that target heart and various tumors as well as to their use for targeting. The present invention also provides a composition comprising at least one therapeutic agent and at least one peptide of the invention or, alternatively, a nucleic acid molecule encoding such a peptide as well as its use for the preparation of a drug intended for gene transfer. The present invention also provides an adenoviral vector comprising at least one peptide of the invention exposed at the surface of the viral particle and its use for targeting tumor cells as well as a method for treating or preventing a cancer or a tumor comprising administering said adenoviral vector.

### CROSS-REFERENCE TO EARLIER FILED/PRIORITY/PCT APPLICATIONS

This application is a Continuation-in-Part of copending application Ser. No. 10/182,573, filed Jul. 31, 2002, and claims the 35 U.S.C. § 120 benefits thereof. This application also claims priority under 35 U.S.C. § 119 of EP/00440030.5, filed Feb. 2, 2000, EP/00440229.3, filed Aug. 21, 2000 and PCT/EP01/00894, filed Jan. 26, 2001, and of domestic provisional applications Serial No. 60/186,760 filed Mar. 3, 2000 and Serial No. 60/246,091, filed Nov. 7, 2000. Each of the above applications is hereby expressly incorporated by reference.

### BACKGROUND OF THE INVENTION

1. Technical Field of the Invention

The present invention provides novel peptides and, more particularly peptides that are able to target preferentially heart and various tumor cells. The present invention also relates to a composition comprising such a peptide and a therapeutic agent. The invention is of very special interest in relation to prospect for gene therapy, in particular in human.

2. Description of the Prior Art

Gene therapy can be defined as the transfer of genetic material into a cell or an organism to treat or prevent a cell deficiency or insufficiency. The possibility of treating human disorders by gene therapy has changed in a few years from the stage of theoretical considerations to that of clinical applications. The first protocol applied to man was initiated in the USA in September 1990 on a patient who was genetically immunodeficient as a result of a mutation affecting the gene encoding adenine deaminase (ADA) and the relative success of this first experiment encouraged the development of the technology for various inherited as well as acquired diseases.

Successful gene therapy depends on the efficient delivery of a therapeutic gene to the cells of a living organism and expression of the genetic information. Functional genes can be introduced into cells by a variety of techniques resulting in either transient expression (transient transfection) or permanent transformation of the host cells with incorporation into the host genome. Whereas direct injection of naked nucleic acids (i.e., plasmidic DNA) can be envisaged (Wolff et al., Science 247 (1990) 1465-1468), the majority of the protocols uses vectors to carry the genes of interest. The vectors can be divided into two categories.

The first category relates to viral vectors, especially adeno- and retroviral vectors. Viruses have developed diverse and highly sophisticated mechanisms to achieve transport across the cellular membrane, escape from lysosomal degradation, delivery of their genome to the nucleus and, consequently, have been used in many gene delivery applications. Their structure, organization and biology are described in the literature available to a person skilled in the art.

One of the most widely used vectors for in vivo gene transfer is a replication-deficient recombinant adenoviral vector. Some of its advantages are the fact that it can be grown to high titers and can efficiently transduce a wide variety of human cell types. The adenoviral genome consists of a linear double-standed DNA molecule of approximately 36 kb carrying more than about thirty genes necessary to complete the viral cycle. The early genes are divided into 4 regions (E1 to E4) that are essential for viral replication with the exception of the E3 region, which is believed to modulate the anti-viral host immune response. The late genes encode in their majority the structural proteins constituting the viral capsid. In addition, the adenoviral genome carries at both extremities cis-acting 5′ and 3′ ITR (Inverted Terminal Repeat) and packaging sequences essential for DNA replication. The adenoviral vectors used in gene therapy protocols lack most of the E1 region in order to avoid their replication and subsequent dissemination in the environment and the host body. Additional deletions in the E3 region increase the cloning capacity (for a review see for example Yeh et al. FASEB Journal 11 (1997) 615-623). Second generation vectors retaining the ITRs and packaging sequences and containing substantial genetic modifications aimed to abolish the residual synthesis of the viral antigens, are currently constructed in order to improve long-term expression of the therapeutic gene in the transduced cells (WO 94/28152, Lusky et al., J. Virol 72 (1998) 2022-2032).

The specificity of infection of the adenoviruses is determined by the attachment of the virions to cellular receptors present at the surface of the permissive cells. In this regard, the fiber present at the surface of the viral capsid play a critical role in cellular attachment (Defer et al., J. Virol. 64 (1990) 3661-3673) and penton-base promotes internalization through the binding to the cellular integrins (Mathias et al., J. Virol. 68 (1994) 6811-6814). Recent studies have presumed the use of the coxsackie virus receptor (CAR) by the types 2 and 5 adenoviruses (Bergelson et al; Science 275 (1997) 1320-1323). However, other surface proteins may be involved in fiber attachment, for example, the 2 domain of the class I histocompatibility antigens as identified by Hong et al., (EMBO J. 16 (1997) 2294-2306). The fiber is composed of 3 regions (Chroboczek et al., Current Top. Microbiol. Immunol. 199 (1995) 165-200): the tail at the N-terminus of the protein which interacts with penton base and ensures the anchorage in the capsid, the shaft composed of a number of -sheets repeats and the knob which contains the trimerization signals (Hong et al., J. Virol. 70 (1996) 7071-7078) and the receptor binding moiety (Henri et al., J. Virol. 68 (1994) 5239-5246; Louis et al., J. Virol. 68 (1994) 4104-4106).

The second category relates to synthetic vectors. A large number derived from various lipids and polymers are currently available (for a review, see for example Rolland, Critical reviews in Therapeutic Drug Carrier Systems 15 (1998) 143-198). Although less efficient than viral delivery systems, they present potential advantages with respect to large-scale production, safety, low immunogenicity and cloning capacity. Moreover, they can be easily modified by simple mixing of the desired components.

The design of viral and synthetic gene therapy vectors which are capable to deliver therapeutic genes to a specific cell represents one of the main interest and challenge in today's gene therapy research. The use of targeting vectors would limit the vector spread, thus increasing therapeutic efficacy for the desired target cells and minimizing potential side effects. Targeting can be achieved by first identifying a suitable address at the cellular surface and then modifying the vectors in such a way that they can recognize this address.

It has been shown that a cell type or a disease affected cell expresses unique cell surface markers. For example, endothelial cells in rapidly growing tumors express cell surface proteins not present in quiescent endothelium, i.e., αv integrins (Brooks et al., Science 264 (1994) 569) and receptors for certain angiogenic growth factors (Hanahan Science 277 (1997) 48). Phage display library selection methods can be employed to select peptide sequences that interact with these particular cell surface markers (see for example U.S. Pat. Nos. 5,622,699, and 5,403,484). In this system, a random peptide is expressed on the phage surface by fusion of the corresponding coding sequence to a gene encoding one of the phage surface proteins. The desired phages are selected on the basis of their binding to the target such as isolated organ fragments (ex vivo procedure) or cultured cells (in vitro procedure). Identification of targeting peptides can also be done by an in vivo procedure that is achieved by injecting phage libraries into mice and subsequently rescuing the bound phages from the targeted organs. Selected peptides are identified by sequencing the genome phage region encoding the displayed peptide. In vivo organ screening was successfully applied to isolate peptide sequences that conferred selective phage homing to the brain and kidney (Pasqualini et al., Nature 380 (1996) 364-366), to the vasculature of lung, skin and pancreas (Rajotte et al., J. Clin. Invest. 102 (1998) 430-437) and to several tumor types (Pasqualini et al., Nature Biotechnology 15 (1997) 542-546).

Furthermore, tumors could be targeted not only via their vasculature but also via the extracellular matrix (ECM) or the tumor cells themselves. Since blood vessels are constantly modified in tumors, the endothelium is locally disrupted allowing gene therapy vectors to extravasate and interact with the ECM and tumor cells. Peptides which interact with the ECM or tumor-associated cell surface markers could also be selected using the phage display technique (Christiano et al., Cancer Gene Therapy 3 (1996) 4-10; Croce et al., Anticancer Res. 17 (1997) 4287-4292; Gottschalk et al. Gene Ther. 1 (1994) 185-191; Park et al. Adv Pharmacol. 40 (1997) 399-435). As an example, a HWGF motif was identified as a ligand of the matrix metalloproteinases involved in tumor growth, angiogenesis and metastasis. Administration of a HWGF comprising peptide to a tumor bearing animal model prevents tumor growth and invasion and prolongs animal survival (Koivunen et al., Nature Biotechnology 17 (1999) 768-774).

Recently, Romanczuk et al., (Human Gene Therapy 10 (1999) 2615-2626) reported the isolation of peptides targeting the differentiated, cilliated airway epithelial cells. Coupling of the best binding peptide to the surface of a recombinant adenovirus with bifunctional polyethylene glycol (PEG) resulted in a vector able to transduce the target cells via an alternative pathway dependent on the incorporated peptide.

### SUMMARY OF THE INVENTION

All together, very few cell type or disease-specific surface markers have been described up to now and only very few ligands are known that specifically interact with such markers. Therefore, the technical problem underlying the present invention is the provision of improved methods and means for the targeting of therapeutic agents to specific cells.

This technical problem is solved by the provision of the embodiments as defined in the claims.

Accordingly, the present invention relates to a peptide selected from the group consisting of:

| | |
|---|---|
| X₁THPRFATX₂ | (SEQ ID NO: 1) |
| X₁RTPFATYX₂ | (SEQ ID NO: 2) |
| X₁FHVNPTSPTHPLX₂ | (SEQ ID NO: 3) |
| X₁QTSSPTPLSHTQX₂ | (SEQ ID NO: 4) |
| X₁PQTSTLLX₂ | (SEQ ID NO: 5) |
| X₁HLPTSSLFDTTHX₂ | (SEQ ID NO: 6) |
| X₁VHHLPRTX₂ | (SEQ ID NO: 7) |
| X₁QLHNHLPX₂ | (SEQ ID NO: 8) |
| X₁HSFDHLPAAALHX₂ | (SEQ ID NO: 9) |
| X₁YPSAPPQWLTNTX₂ | (SEQ ID NO: 10) |
| X₁YPSQSQRX3LSX4HX₂ | (SEQ ID NO: 11) |
| X₁TYPSSTLX₂ | (SEQ ID NO: 12) |
| X₁NTLQVRGVYPSVX₂ | (SEQ ID NO: 13) |
| X₁YSNRTNTNSHWAX₂ | (SEQ ID NO: 14) |
| X₁PATNTSKX₂ | (SEQ ID NO: 15) |
| X₁HVNKLHGX₂ | (SEQ ID NO: 16) |
| X₁FHVNPTSPTHPLX₂ | (SEQ ID NO: 17) |
| X₁NANKLWTWVSSPX₂ | (SEQ ID NO: 18) |
| X₁SGRIPYLX₂ | (SEQ ID NO: 19) |
| X₁NEDINDVSGRLSX₂ | (SEQ ID NO: 20) |
| X₁LSPQRASQRLYSX₂ | (SEQ ID NO: 21) |
| X₁SFSTSPQX₂ | (SEQ ID NO: 22) |
| X₁ERMDSPQX₂ | (SEQ ID NO: 23) |
| X₁HHGHSPTSPQVRX₂ | (SEQ ID NO: 24) |
| X₁GSSTGPQRLHVPX₂ | (SEQ ID NO: 25) |
| X₁TCSLCNPVQPQRX₂ | (SEQ ID NO: 26) |
| X₁QRLTTLYX₂ | (SEQ ID NO: 27) |
| X₁WSPGQQRLHNSTX₂ | (SEQ ID NO: 28) |
| X₁WKSELPVQRARFX₂ | (SEQ ID NO: 29) |
| X₁SELPSMRLYTQPX₂ | (SEQ ID NO: 30) |
| X₁HSLHVHKGLSELX₂ | (SEQ ID NO: 31) |
| X₁SDLPVQLEPERQX₂ | (SEQ ID NO: 32) |
| X₁TRYLPVLPSLFPX₂ | (SEQ ID NO: 33) |
| X₁TCSLCNPVQPQRX₂ | (SEQ ID NO: 34) |
| X₁WEPPVQSAWQLSX₂ | (SEQ ID NO: 35) |
| X₁HFTFPQQQPPRPX₂ | (SEQ ID NO: 36) |
| X₁GSTSRPQPPSTVX₂ | (SEQ ID NO: 37) |
| X₁NFSQPPSKHTRSX₂ | (SEQ ID NO: 38) |
| X₁QYPHKYTLQPPKX₂ | (SEQ ID NO: 39) |
| X₁FNQPPSWRVSNSX₂ | (SEQ ID NO: 40) |
| X₁SVSVGMKPSPRPX₂ | (SEQ ID NO: 41) |
| X₁STPRPPLGIPAQX₂ | (SEQ ID NO: 42) |
| X₁TQSPLNYRPALLX₂ | (SEQ ID NO: 43) |
| X₁AQSPTIKLTPSWX₂ | (SEQ ID NO: 44) |
| X₁HNLLTQSX₂ | (SEQ ID NO: 45) |
| X₁TLVQSPMX₂ | (SEQ ID NO: 46) |
| X₁NLNTDNYRQLRHX₂ | ((SEQ ID NO: 47) |
| X₁FRPAVHNMPSLQX₂ | (SEQ ID NO: 48) |
| X₁ISRPAPISVDMKX₂ | (SEQ ID NO: 49) |
| X₁THRPSLPDSSRAX₂ | (SEQ ID NO: 50) |
| X₁ALHPLTHRHYATX₂ | (SEQ ID NO: 51) |
| X₁THRGPQSX₂ | (SEQ ID NO: 52) |
| X₁SFHMPSRAVSLSX₂ | (SEQ ID NO: 53) |
| X₁NQSNFTSRALLYX₂ | (SEQ ID NO: 54) |
| X₁SFPTHIDHHVSPX₂ | (SEQ ID NO: 55) |
| X₁LNGDPTHX₂ | (SEQ ID NO: 56) |
| X₁HMPHHVSNLQLHX₂ | (SEQ ID NO: 57) |
| X₁LPSVSPVLQVLGX₂ | (SEQ ID NO: 58) |
| X₁DAQQLYLSNWRSX₂ | (SEQ ID NO: 59) |
| X₁DSYLSSTLPGQLX₂ | (SEQ ID NO: 60) |
| X₁SPTPTSHQQLHSX₂ | (SEQ ID NO: 61) |
| X₁APPGNWRNYLMPX₂ | (SEQ ID NO: 62) |
| X₁LSNKMSQX₂ | (SEQ ID NO: 63) |
| X₁MHNVSDSNDSAIX₂ | (SEQ ID NO: 64) |
| X₁DNSNDLMX₂ | (SEQ ID NO: 65) |
| X₁TVMEAPRSAILIX₂ | (SEQ ID NO: 66) |
| X₁CNDIGWVRCX₂ | (SEQ ID NO: 67) |
| X₁CWPYPSHFCX₂ | (SEQ ID NO: 68) |
| X₁MPLPQPSHLPLLX₂ | (SEQ ID NO: 69) |
| X₁LPQRAFWVPPIVX₂ | (SEQ ID NO: 70) |
| X₁WPVRPWMPGPVVX₂ | (SEQ ID NO: 71) |
| X₁WPTSPWLEREPAX₂ | (SEQ ID NO: 72) |
| X₁WPTSPWSSRDWSX₂ | (SEQ ID NO: 73) |
| X₁HEWSYLAPYPWFX₂ | (SEQ ID NO: 74) |
| X₁QIDRWFDAVQWLX₂ | (SEQ ID NO: 75) |
| X₁CLPSTRWTCX₂ | (SEQ ID NO: 76) |
| X₁CWPMKSX₅FCX₂ | (SEQ ID NO: 77) |

wherein each X₁ and X₂ independently of one another represents any amino acid sequence of n amino acids, n varying from 0 to 50 and n being identical or different in X₁ and X₂, and wherein X₃, X₄ and X₅, identical or different, represent any amino acid. Preferably, X₅ is a leucine (L) or a glutamine (Q) residue.

These peptides are useful to direct e.g., gene therapy vectors to specific targets in an organism.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

The term [symbol] amino acid [symbol] and residues are synonyms. This term refers to natural, unnatural and/or synthetic amino acids, including D or L optical isomers, modified amino acids and amino acid analogs.

The terms [symbol] peptide [symbol] and [symbol] amino acid [symbol] used herein are intended to have the same meaning as commonly understood by one of ordinary skill in the art. According to a preferred embodiment, n is ranging independently of one another in X₁ and X₂ from 0 to about 10 amino acids and more preferably from 0 to about 5 amino acids. Peptides according to the invention may be produced de novo by synthetic methods or by expression of the appropriate DNA fragment by recombinant DNA techniques in eukaryotic as well as prokaryotic cells. Alternatively, they can also be produced by fusion to a fusion partner. When the fusion partner is a polypeptide, fusion can be designed to place the peptide at the N- or C terminus or between two residues of said polypeptide.

### DETAILED DESCRIPTION OF BEST MODE AND SPECIFIC/PREFERRED EMBODIMENTS OF THE INVENTION

The peptide according to the invention can be purified by art known techniques such as reverse phase chromatography, size exclusion, high performance liquid chromatography, ion exchange chromatography, gel electrophoresis, affinity chromatography and the like. The conditions and technology used to purify a particular peptide of the invention will depend on the synthesis method and on factors such as net charge, hydrophobicity, hydrophilicity and will be apparent to those having skill in the art.

Optionally, the peptide of the invention may include modifications of one or more amino acid residue(s) by way of substitution or addition of moieties (i.e., glycosylation, alkylation, acetylation, amidation, phosphorylation and the like). Included within the scope of the present invention are for example peptides containing one or more analogs of an amino acid (including not naturally occurring amino acids), peptides with substituted linkages as well as other modifications known in the art both naturally occurring and non naturally occurring. The peptide can be linear or cyclized for example by flanking the peptide at both extremities by cysteine residues. In accordance with the aim pursued with the present invention, preferred modifications are those that allow or improve the coupling of a peptide of the invention to a therapeutic agent as described hereinafter (i.e., addition of sulfhydryl, amine groups . . . ). The present invention also encompasses analogs of a peptide according to the invention where at least one amino acid is replaced by another amino acid having similar properties. The matrix of FIGS. 84 and 85 of Atlas of Protein Sequence and Structure (1978, Vol. 5, ed. M. O. Dayhoff, National Biomedical Research Foundation, Washington, D.C.) show the groups of chemically similar amino acids that tend to replace one another: the hydrophobic group; the aromatic group; the basic group; the acid, acid-amide group; cysteine; and the other hydrophilic residues. Analogs can also be retro or inverso peptides (WO 95/24916).

The present invention also contemplates modifications that render the peptides of the invention detectable. For this purpose, the peptides of the invention can be modified with a detectable moiety (i.e., a scintigraphic, radioactive, fluorescent, or dye labels and the like). Suitable radioactive labels include but are not limited to Tc^{99m}, I¹²³ and In¹¹¹. Such labels can be attached to the peptide of the invention in known manner, for example via a cysteine residue. Other techniques are described elsewhere.

The peptides of the invention may be used for a variety of purposes.

According to a first and preferred alternative (second embodiment), a peptide of the invention may be used for targeting purposes. Targeting is defined as the capability of recognizing and binding preferentially to a cell intended to be targeted. [symbol] Preferentially [symbol] means that the peptide of the invention provides lesser attachment to a non target cell compared to a target cell. Generally, a particular peptide of the invention recognizes and binds a marker that is expressed or exposed at the surface of such a cell (i.e., cell surface marker, receptor, peptide presented by the histocompatibility antigens, tumor-specific antigen . . . ). Within the framework of the present invention, it may be advantageous to target more particularly a tumor cell, a particular cell type or a category of cells. Examples of particular cell types include but are not limited to liver and heart cells. Categories of cells include cells of artherosclerotic plaques, ischaemic regions, parenchyme, ECM, vasculature, coronary artery.

A second alternative is a use related to the study, isolation and purification of the cell surface markers to which such peptides specifically bind. Another alternative relates to diagnostic purposes for example for imaging the target cells exhibiting such markers by in vitro as well as in vivo assays. Accordingly, the scope of the present invention also includes a diagnostic reagent for detection of a target cell, said reagent comprising a peptide according to the invention and a carrier. Preferably, the peptide is modified with a detectable moiety and the carrier is for systemic injection.

Finally, a peptide according to the invention may be used for therapeutic as well as prophylactic purposes, intended for the treatment of the human or animal body. A peptide according to the present invention may have therapeutic effects by itself (i.e., angiostatic, inhibitors of metalloproteases, cell-cycle inhibitors, cytostatic, cytotoxic, endosome reduction, membranolytic, proliferation-inducing properties . . . ) in addition to its targeting properties (see for example Koivunen et al., Nat. Biotech. 17 (1999) 768-774).

According to a third embodiment, the present invention also provides peptides for heart targeting. A heart targeting peptide of the invention has a minimal size of 7 amino acids. Such peptides can be classified in different families that are defined according to the presence of some common amino acid motifs. Each peptide in a family contains a particular motif but in a different amino acid environment. The present invention also encompasses the case where a particular peptide comprises more than one selected motif that can be continuous, separated by a stretch of residues or overlapping. X₁, X₂, X₃, X₄ and n are as defined above.

Such peptides can be used for the targeting specifically to heart muscle and are more specifically intended for muscular dystrophy, heart diseases or coronary heart diseases. Systemic delivery of vectors targeted with such heart-specific peptides can be considered to avoid regional delivery to the coronary artery that requires an invasive and cumbersome operation. Alternatively, the use of such targeting peptides will limit the spread of vectors after local administration.

A first family relates to a heart targeting peptide comprising at least a three amino acid motif THP or FAT or THP and FAT. Advantageously, it comprises both the THP and FAT motifs, especially when the two motifs are separated by at least one amino acid. Preferably, a heart targeting peptide according to the invention has the sequence:

| | |
|---|---|
| X₁THPRFATX₂, | (SEQ ID NO: 1) |
| X₁RTPFATYX₂, or | (SEQ ID NO: 2) |
| X₁FHVNPTSPTHPLX₂. | (SEQ ID NO: 3) |

A second family relates to a heart targeting peptide comprising at least a three amino acid motif QTS. Preferably a heart targeting peptide according to the invention has the sequence:

| | |
|---|---|
| X₁QTSSPTPLSHTQX₂ or | (SEQ ID NO: 4) |
| X₁PQTSTLLX₂. | (SEQ ID NO: 5) |

A third family relates to a heart targeting peptide comprising at least a three amino acid motif HLP or SLF or HLP and SLF. Advantageously, it comprises both the HLP and SLF motifs, especially when the two motifs are separated by at least one amino acid. Preferably, a heart targeting peptide according to the invention has the sequence:

| | |
|---|---|
| X₁HLPTSSLFDTTHX₂, | (SEQ ID NO: 6) |
| X₁VHHLPRTX₂, | (SEQ ID NO: 7) |
| X₁QLHNHLPX₂, | (SEQ ID NO: 8) |
| X₁HSFDHLPAAALHX₂, or | (SEQ ID NO: 9) |
| X₁TRYLPVLPSLFPX₂. | (SEQ ID NO: 33) |

A fourth family relates to a heart targeting peptide comprising at least a three amino acid motif YPS or TNT or YPS and TNT. Advantageously, it comprises both the YPS and TNT motifs, especially when the two motifs are separated by three to eight amino acids. Preferably, a heart targeting peptide according to the invention has the sequence:

| | |
|---|---|
| X₁YPSAPPQWLTNTX₂, | (SEQ ID NO: 10) |
| X₁YPSQSQRX₃LSX₄HX₂, | (SEQ ID NO: 11) |
| X₁TYPSSTLX₂, | (SEQ ID NO: 12) |
| X₁NTLQVRGVYPSVX₂, | (SEQ ID NO: 13) |
| X₁YSNRTNTNSHWAX₂, or | (SEQ ID NO: 14) |
| X₁PATNTSKX₂. | (SEQ ID NO: 15) |

A fifth family relates to a heart targeting peptide comprising at least a three amino acid motif HVN or NKL or HVN and NKL. Advantageously, it comprises both HVN and NKL motifs, especially when the two motifs are overlapping. Preferably, a heart targeting peptide of the invention has the sequence:

| | |
|---|---|
| X₁HVNKLHGX₂, | (SEQ ID NO: 16) |
| X₁FHVNPTSPTHPLX₂, or | (SEQ ID NO: 17) |
| X₁NANKLWTWVSSPX₂. | (SEQ ID NO: 18) |

A sixth family relates to a heart targeting peptide comprising at least a three amino acid motif SGR. Preferably, a heart targeting peptide according to the invention has the sequence:

| | |
|---|---|
| X₁SGRIPYLX₂, or | (SEQ ID NO: 19) |
| X₁NEDINDVSGRLSX₂. | (SEQ ID NO: 20) |

A seventh family relates to a heart targeting peptide comprising at least a three amino acid motif SPQ, QRA, QRL or PQR or any combination thereof. Advantageously, it comprises the three motifs SPQ, QRA and QRL, especially when the SPQ and QRA motifs are overlapping and separated from the QRL motif by at least one amino acid. Preferably, a heart targeting peptide according to the present invention has the sequence:

| | |
|---|---|
| X₁LSPQRASQRLYSX₂, | (SEQ ID NO: 21) |
| X₁SFSTSPQX₂, | (SEQ ID NO: 22) |
| X₁ERMDSPQX₂, | (SEQ ID NO: 23) |
| X₁WKSELPVQRARFX₂, | (SEQ ID NO: 29) |
| X₁HHGHSPTSPQVRX₂, | (SEQ ID NO: 24) |
| X₁GSSTGPQRLHVPX₂, | (SEQ ID NO: 25) |
| X₁YPSQSQRX₃LSX₄HX₂, | (SEQ ID NO: 11) |
| X₁TCSLCNPVQPQRX₂, | (SEQ ID NO: 26) |
| X₁QRLTTLYX₂, or | (SEQ ID NO: 27) |
| X₁WSPGQQRLHNSTX₂. | (SEQ ID NO: 28) |

An eighth family relates to a heart targeting peptide comprising at least a three amino acid motif SEL or PVQ or SEL and PVQ. Advantageously, it comprises both the SEL and PVQ motifs, especially when the two motifs are continuous. Preferably, a heart targeting peptide according to the invention has the sequence:

| | |
|---|---|
| X₁WKSELPVQRARFX₂, | (SEQ ID NO: 29) |
| X₁SELPSMRLYTQPX₂, | (SEQ ID NO: 30) |
| X₁HSLHVHKGLSELX₂, | (SEQ ID NO: 31) |
| X₁SDLPVQLEPERQX₂, | (SEQ ID NO: 32) |
| X₁TCSLCNPVQPQRX₂, or | (SEQ ID NO: 34) |
| X₁WEPPVQSAWQLSX₂. | (SEQ ID NO: 35) |

A ninth family relates to a heart targeting peptide comprising at least a three amino acid motif QPP or PRP or QPP and PRP. Advantageously, it comprises both the QPP and PRP motifs, especially when the two motifs are continuous. Preferably, a heart targeting peptide according to the invention has the sequence:

| | |
|---|---|
| X₁HFTFPQQQPPRPX₂, | (SEQ ID NO: 36) |
| X₁GSTSRPQPPSTVX₂, | (SEQ ID NO: 37) |
| X₁NFSQPPSKLITRSX₂, | (SEQ ID NO: 38) |
| X₁QYPHKYTLQPPKX₂, | (SEQ ID NO: 39) |
| X₁FNQPPSWRVSNSX₂, | (SEQ ID NO: 40) |
| X₁SVSVGMKPSPRPX₂, or | (SEQ ID NO: 41) |
| X₁STPRPPLGIPAQX₂. | (SEQ ID NO: 42) |

Heart targeting peptides of the invention are more advantageously intended for targeting any heart cells including the heart vasculature, especially endothelial cells, and heart muscle cells.

According to a fourth embodiment, the present invention provides tumor targeting peptides. A tumor targeting peptide according to the invention has a minimal size of 7 amino acids. Such peptides can be classified in different families that are defined according to the presence of some common amino acid motifs. Each peptide in a family contains a particular motif but in a different amino acid environment. The present invention also encompasses the case where a particular peptide comprises more than one selected motif, that can be continuous, separated by a stretch of residues or overlapping. X₁, X₂ and n are as defined above.

Coupling of these peptides to plasmids, viral and synthetic vectors will, for example, allows after systemic administration the targeting of tumor metastasis or tumor sites that are difficult to reach surgically. Alternatively, local administration can also be envisaged with the advantage of limiting the spread of vectors.

A first family relates to a tumor targeting peptide comprising at least a three amino acid motif RPA, NYR or QSP or any combination thereof. Advantageously, it comprises the three motifs RPA, NYR and QSP, especially when the QSP motif is separated from the NYR motif by at least one amino acid and the NYR and RPA motifs are overlapping. Preferably, a tumor targeting peptide according to the invention has the sequence:

| | |
|---|---|
| X₁TQSPLNYRPALLX₂, | (SEQ ID NO: 43) |
| X₁AQSPTIKLTPSWX₂, | (SEQ ID NO: 44) |
| X₁TLVQSPMX₂, | (SEQ ID NO: 46) |
| X₁NLNTDNYRQLRHX₂, | (SEQ ID NO: 47) |
| X₁FRPAVHNMPSLQX₂, or | (SEQ ID NO: 48) |
| X₁SRPAPISVDMKX₂. | (SEQ ID NO: 49) |

A second family relates to a tumor targeting peptide comprising at least a three amino acid motif THR or SRA or THR and SRA. Advantageously, it comprises both the THR and SRA motifs, especially when the two motifs are separated by four to eight amino acids. Preferably, a tumor targeting peptide according to the invention has the sequence:

| | |
|---|---|
| X₁THRPSLPDSSRAX₂, | (SEQ ID NO: 50) |
| X₁ALHPLTHRHYATX₂, | (SEQ ID NO: 51) |
| X₁THRGPQSX₂, | (SEQ ID NO: 52) |
| X₁SFHMPSRAVSLSX₂, or | (SEQ ID NO: 53) |
| X₁NQSNFTSRALLYX₂. | (SEQ ID NO: 54) |

A third family relates to a tumor targeting peptide comprising at least a three amino acid motif PTH, VSP or a four amino acid motif HHVS or any combination thereof. Advantageously, it comprises the three motifs PTH, HHVS and VSP, especially when the PTH motif is separated from the HHVS motif by at least one amino acid and the HHVS and VSP motifs are overlapping. Preferably, a tumor targeting peptide according to the invention has the sequence:

| | |
|---|---|
| X₁SFPTHIDHHVSPX₂, | (SEQ ID NO: 55) |
| X₁LNGDPTHX₂, | (SEQ ID NO: 56) |
| X₁HMPHHVSNLQLHX₂ or | (SEQ ID NO: 57) |
| X₁LPSVSPVLQVLGX₂. | (SEQ ID NO: 58) |

A fourth family relates to a tumor targeting peptide comprising at least a three amino acid motif YLS or QQL or YLS and QQL. Advantageously, it comprises both YLS and QQL motifs, especially when the two motifs are continuous. Preferably a tumor targeting peptide according to the invention has the sequence:

| | |
|---|---|
| X₁DAQQLYLSNWRSX₂, | (SEQ ID NO: 59) |
| X₁DSYLSSTLPGQLX₂ or | (SEQ ID NO: 60) |
| X₁SPTPTSHQQLHSX₂. | (SEQ ID NO: 61) |

A fifth family relates to a tumor-targeting peptide comprising at least a three amino acid motif SND or SAI or SND and SAI. Advantageously, it comprises both the SND and SAI motifs, especially when the two motifs are continuous. Preferably, a tumor targeting peptide according to the invention has the sequence:

| | |
|---|---|
| X₁MHNVSDSNDSAIX₂, | (SEQ ID NO: 64) |
| X₁DNSNDLMX₂, or | (SEQ ID NO: 65) |
| X₁TVMEAPRSAILIX₂. | (SEQ ID NO: 66) |

A sixth family relates to a tumor-targeting peptide comprising at least a three amino acid motif NDI, WPY, MPL, PSH, LPQ, WPV or WPT or any combination thereof. According to a special embodiment, said sixth family relates to a said peptide comprising at least one amino acid motif WPX₃X₄PW, with X₃ and X₄, identical or different, represent any amino acid; preferably X₃ is V or T and/or X₄ is R or S. In another special embodiment, said peptide comprises at least one amino acid motif WPTSPWX₃X₄RX₅ with X₃, X₄ and X₅, identical or different, represent any amino acid; preferably X₃ is L or S and/or X₄ is E or S and/or X₅ is E or D. In another special embodiment, said peptide comprises at least one amino acid motif WPX₃X₄SX₅F with X₃, X₄ and X₅, identical or different, represent any amino acid; preferably X₃ is Y or M and/or X₄ is P or K and/or X₅ is L, Q or H. Preferably, a tumor targeting peptide according to the invention has the sequence:

| | |
|---|---|
| X₁CNDIGWVRCX₂, | (SEQ ID NO: 67) |
| X₁CWPYPSHFCX₂, | (SEQ ID NO: 68) |
| X₁MPLPQPSHLPLLX₂, | (SEQ ID NO: 69) |
| X₁LPQRAFWVPPIVX₂, | (SEQ ID NO: 70) |
| X₁WPVRPWMPGPVVX₂, | (SEQ ID NO: 71) |
| X₁WPTSPWLEREPAX₂, | (SEQ ID NO: 72) |
| or | |
| X₁WPTSPWSSRDWSX₂. | (SEQ ID NO: 73) |

A seventh family relates to a tumor-targeting peptide comprising at least a three amino acid motif HEW, QID, WPM or CLP or any combination thereof. Preferably, a tumor targeting peptide according to the invention has the sequence:

| | |
|---|---|
| X₁HEWSYLAPYPWFX₂ | (SEQ ID NO: 74) |
| X₁QIDRWFDAVQWLX₂ | (SEQ ID NO: 75) |
| X₁CLPSTRWTCX₂, | (SEQ ID NO: 76) |
| or | |
| X₁CWPMKSX₅FCX₂ | (SEQ ID NO: 77) |

Preferably, X₅ is a leucine (L) or a glutamine (Q) residue.

Advantageously, a tumor targeting peptide of the present invention may be used for the targeting of a therapeutic agent to a tumor cell, a metastasis or a tumor vasculature.

According to a fifth embodiment, the present invention provides for a composition comprising at least one peptide according to the present invention and at least one therapeutic agent or alternatively at least one nucleic acid molecule encoding a peptide of the invention and at least one therapeutic agent.

As used herein, a “therapeutic agent” is used broadly to mean an organic chemical such as a drug (i.e., a cytotoxic drug), a peptide including a variant or a modified peptide or a peptide-like molecule, a protein, an antibody or a fragment thereof such as a Fab (ab for antigen binding), a F(ab′)₂, a Fc (c for crystallisable) or a scFv (sc for single chain and v for variable). Antibody fragments are described in detail in immunology manuals (such as Immunology, third edition 1993, Roitt, Brostoff and Male, ed Gambli, Mosby). It is also possible to use a chimeric antibody or protein derived from the sequence of diverse origins. As an example, humanized antibodies combine part of the variable regions of a mouse antibody and constant regions of a human immunoglobulin. Within the context of the present invention, a protein is more preferably an immunostimulatory protein, such as B7.1, B7.2, CD40, ICAM, CD4, CD8 and the like. A therapeutic agent may also be a nucleic acid molecule e.g. DNA, or RNA, antisense or sense, oligonucleotide, double-stranded or single-stranded, circular or linear . . . etc.

In a preferred embodiment, a therapeutic agent is a vector for delivering at least one therapeutic gene or gene of interest to a target cell of a vertebrate. In the context of the present invention, it can be a plasmid, a synthetic (non viral) or a viral vector.

Plasmid denotes an extrachromosomic circular DNA capable of autonomous replication in a given cell. The choice of the plasmids is very large. It is preferably designed for amplification in bacteria and expression in eukaryotic host cell. Such plasmids can be purchased from a variety of manufacturers. Suitable plasmids include but are not limited to those derived from pBR322 (Gibco BRL), pUC (Gibco BRL), pBluescript (Stratagene), pREP4, pCEP4 (Invitrogene), pCI (Promega) and p Poly (Lathe et al., Gene 57 (1987), 193-201). It is also possible to engineer such a plasmid by molecular biology techniques (Sambrook et al., Laboratory Manual, Cold Spring Harbor Laboratory Press, Cold Spring Harbor (1989), NY). A plasmid may also comprise a selection gene in order to select or identify the transfected cells (e.g., by complementation of a cell auxotrophy, antibiotic resistance), stabilizing elements (e.g. cer sequence; Summers and Sherrat, Cell 36 (1984), 1097-1103) or integrative elements (e.g., LTR viral sequences).

A vector may also be from viral origin and may be derived from a variety of viruses, such as herpes viruses, cytomegaloviruses, foamy viruses, lentiviruses, AAV (adeno-associated virus), poxviruses, adenoviruses and retroviruses. Such viral vectors are well known in the art. The term [symbol] viral vector [symbol] as used in the present invention encompasses the vector genome, the viral particles (i.e., the viral capsid including the viral genome) as well as empty viral capsids.

A viral vector which is particularly appropriate for the present invention is an adenoviral vector (for a review see for example Hitt et al., Advances in Pharmacology 40 (1997) 137-206). In one embodiment, the adenoviral vector is engineered to be conditionally replicative (CRAd vectors) in order to replicate selectively in specific cells (e.g., proliferative cells) as described in Heise and Kirn (2000, J. Clin. Invest. 105, 847-851). According to a second and preferred alternative, it is replication-defective, especially for E1 functions by total or partial deletion of the respective region. Advantageously, the E1 deletion covers nucleotides (nt) 458 to 3510 by reference to the sequence of the human adenovirus type 5 disclosed in the Genebank data base under the reference M 73260. Furthermore, the adenoviral backbone of the vector may comprise additional modifications, such as deletions, insertions or mutations in one or more viral genes. In this respect, the adenoviral vector can be a multiply deficient adenoviral vector having a deficiency in one or more essential gene functions of the E1 region and a deficiency in one or more essential gene functions in either or both of the E2 and the E4 region. It is preferred in the context of the invention, that such a deficient adenoviral vector has a deficiency in one or more essential gene functions of the E2A region. An example of an E2 modification is illustrated by the thermosensible mutation located on the DBP (DNA Binding Protein) encoding gene (Ensinger et al., J. Virol. 10 (1972), 328-339). The adenoviral vector may also be deleted of all or part of the E2 region, including either or both of the early E2A and the early E2B region, with a special preference for deletion within the E2A region (coding sequence and/or promoter) or of all the E2A region The adenoviral sequence may also be deleted of all or part of the E4 region. A partial deletion retaining the ORFs 3 and 4 or ORFs 3 and 6/7 may be advantageous (see for example European patent application EP-98401722.8). In addition, the adenoviral vector in use in the present invention may be deleted of all or part of the E3 region. In this context, it might be interesting to retain the E3 sequences coding for the polypeptides allowing to escape the host immune system (Gooding et al., Critical Review of Immunology 10 (1990), 53-71). A defective adenoviral vector deficient in all early and late regions may also be envisaged. Any one of the deleted functional genes/regions then may be replaced with a gene of interest placed under the control of the necessary elements to permit its expression in a host cell.

The adenoviral vector in use in the present invention may be derived from a human or animal adenovirus genome, in particular a canine, avian, bovine, murine, ovine, feline, porcine or simian adenovirus or alternatively from a hybrid thereof. Any serotype can be employed. One can cite in particular the canine CAV-1 or CAV-2 adenovirus (Genbank ref CAV1GENOM and CAV77082 respectively), the avian adenovirus (Genbank ref AAVEDSDNA), the mouse adenovirus (Genbank ref ADRMUSMAV1) and the bovine BAV3 (Seshidhar Reddy et al., J. Virol. 72 (1998) 1394-1402). However, the human adenoviruses of C sub-group are preferred and especially adenoviruses 2 (Ad2) and 5 (Ad5). Generally speaking, the cited viruses are available in collections such as ATCC and have been the subject of numerous publications describing their sequence, organization and biology, allowing the artisan to practice them.

The recombinant adenoviral vector is packaged to constitute infectious virions capable of infecting target cells and transferring the therapeutic gene. Infectious adenoviral particles may be prepared according to any conventional technique in the field of the art (e.g., cotransfection of suitable adenoviral fragments in a 293 cell line as described in Graham and Prevect, Methods in Molecular Biology, Vol 7 (1991), Gene Transfer and Expression Protocols; Ed E. J. Murray, The Human Press Inc, Clinton, N.J.; homologous recombination as described in WO 96/17070). The defective virions are usually propagated in a complementation cell line or via a helper virus, which supplies in trans the non functional viral genes. The cell line 293 is commonly used to complement the E1 function (Graham et al., J. Gen. Virol. 36 (1977), 59-72) as well as the PER-C6 cells (Fallaux et al., Human Gene Ther. 9 (1998), 1909-1917). Other cell lines have been engineered to complement doubly defective vectors (Yeh et al., J. Virol. 70 (1996), 559-565; Krougliak and Graham, Human Gene Ther. 6 (1995), 1575-1586; Wang et al., Gene Ther. 2 (1995), 775-783; Lusky et al., J. Virol. 72 (1998), 2022-2033; WO 94/28152 and WO 97/04119). The infectious viral particles may be recovered from the culture supernatant but also from the cells after lysis and optionally further purified according to standard techniques (chromatography, ultracentrifugation in a cesium chloride gradient . . . ; see for example WO 96/27677, WO 98/00524, WO 98/22588, WO 98/26048, WO 00/40702, EP-1,016,700 and WO 00/50573).

In addition, adenoviral virions or empty adenoviral capsids can also be used to transfer nucleic acids (i.e., plasmidic vectors) by a virus-mediated cointernalization process as described in U.S. Pat. No. 5,928,944. This process can be accomplished in the presence of a cationic agent(s) such as polycarbenes or lipoplex vesicles comprising one or more lipid layers.

A retroviral vector is also suitable. The numerous vectors described in the literature may be used within the framework of the present invention and especially those derived from murine leukemia viruses (i.e., Moloney or Friend's). Generally, a retroviral vector is deleted of all or part of the viral genes gag, pol and env and comprises 5′LTR, an encapsidation sequence and 3′LTR. These elements may be modified to increase expression level or stability of the retroviral vector. The therapeutic gene is preferably inserted downstream of the encapsidation sequence. The propagation of such a vector requires the use of complementation lines as described in the prior art.

A poxviral vector may be derived e.g., from an avian poxvirus such as the canarypox, a fowlpox virus or a vaccinia virus, the latter being preferred. Among all the vaccinia viruses which can be envisaged within the framework of the present invention, the Copenhagen, Wyeth and modified Ankara (MVA) strains are preferably chosen. The general conditions for obtaining a vaccinia virus capable of expressing a therapeutic gene are disclosed in European patent EP-83, 286 and application EP-206,920. MVA viruses are more particularly described in Mayr et al., (Infection 3 (1975) 6-14) and Sutter and Moss (Proc. Natl. Acad. Sci. USA 89 (1992) 10847-10851).

According to another alternative, a therapeutic agent also refers to a non viral (synthetic) vector that is capable to deliver a therapeutic gene to a target cell, for example lipoplexes. Lipoplexes may contain cationic lipids which have a high affinity for nucleic acids and interact with the cell membranes (Felgner et al., Nature 337 (1989) 387-388). As a result, they are capable of complexing the nucleic acid, thus generating a compact particle capable to enter the cells. Many laboratories have already disclosed various lipoplexes. By way of examples, there may be mentioned DOTMA Felgner et al., Proc. Natl. Acad. Sci. USA 84 (1987), 7413-7417), DOGS or Transfectam™ (Behr et al., Proc. Natl. Acad. Sci. USA 86 (1989), 6982-6986), DMRIE or DORIE (Felgner et al., Methods 5 (1993), 6775), DC-CHOL (Gao and Huang, BBRC 179 (1991), 280-285), DOTAP™ (McLachlan et al., Gene Therapy 2 (1995), 674-622), Lipofectamine™ and glycerolipid compounds (see WO 98/34910 and WO 98/37916).

Other non viral (synthetic) vectors have been developed which are based on cationic polymers such as polyamidoamine (Haensler and Szoka, Bioconjugate Chem. 4 (1993), 372-379), dendritic polymer (WO 95/24221), polyethylene imine or polypropylene imine (WO 96/02655), polylysine (U.S. Pat. No. 5,595,897 or FR-2,719,316), chitosan (U.S. Pat. No. 5,744,166) or DEAE dextran (Lopata et al., Nucleic Acid Res. 12 (1984) 5707-5717).

The term “therapeutic gene or gene of interest” refers to a nucleic acid (DNA, RNA or other polynucleotide derivatives). It can code, e.g., for an antisense RNA, a ribozyme or a messenger (mRNA) that will be translated into a polypeptide. It includes genomic DNA, cDNA or mixed types (minigene). It may code for a mature polypeptide, a precursor (e.g., a precursor comprising a signal sequence intended to be secreted or a precursor intended to be further processed by proteolytic cleavage . . . ), a truncated polypeptide or a chimeric polypeptide. The gene may be isolated from any organism or cell by the conventional techniques of molecular biology (PCR, cloning with appropriate probes, chemical synthesis) and if needed its sequence may be modified by mutagenesis, PCR or any other protocol.

The following genes are of particular interest. For example genes coding for a cytokine (α, β, or γ interferon, interleukine (IL), in particular IL-2, IL-6, IL-10 or IL-12, a tumor necrosis factor (TNF), a colony stimulating factor GM-CSF, C-CSF, M-CSF . . . ), a immunostimulatory polypeptide (B7.1, B7.2, CD40, CD4, CD8, ICAM and the like), a cell or nuclear receptor, a receptor ligand (fas ligand), a coagulation factor (FVIII, FIX . . . ), a growth factor (Transforming Growth Factor TGF, Fibroblast Growth Factor FGF and the like), an enzyme (urease, renin, thrombin, metalloproteinase, nitric oxide synthase NOS, SOD, catalase . . . ), an enzyme inhibitor (α1-antitrypsine, antithrombine III, viral protease inhibitor, plasminogen activator inhibitor PAI-1), the CFTR protein, insulin, dystrophin, a MHC antigen (Major Histocompatibility Complex) of class I or II or a polypeptide that can modulate/regulate expression of cellular genes, a polypeptide capable of inhibiting a bacterial, parasitic or viral infection or its development (antigenic polypeptides, antigenic epitopes, transdominant variants inhibiting the action of a native protein by competition . . . ), an apoptosis inducer or inhibitor (Bax, Bcl2, BclX . . . ), a cytostatic agent (p21, p 16, Rb . . . ), an apolipoprotein (ApoAI, ApoAIV, ApoE . . . ), an inhibitor of angiogenesis (angiostatin, endostatin . . . ), an angiogenic polypeptide (family of Vascular Endothelial Growth Factors VEGF, FGF family, CCN family including CTGF, Cyr61 and Nov), an oxygen radical scaveyer, a polypeptide having an anti-tumor effect, an antibody, a toxin, an immunotoxin and a marker (β-galactosidase, luciferase . . . ) or any other genes of interest that are recognized in the art as being useful for the treatment or prevention of a clinical condition.

In view of treating an hereditary dysfunction, one may use a functional allele of a defective gene, for example a gene encoding factor VIII or IX in the context of haemophilia A or B, dystrophin (or minidystrophin) in the context of myopathies, insulin in the context of diabetes, CFTR (Cystic Fibrosis Transmembrane Conductance Regulator) in the context of cystic fibrosis. Suitable anti-tumor genes include but are not limited to those encoding an antisense RNA, a ribozyme, a cytotoxic product such as thymidine kinase of herpes-I simplex virus (TK-HSV-1), ricin, a bacterial toxin, the expression product of yeast genes FCY1 and/or FUR1 having UPRTase (Uracile Phosphoribosyltransferase) and CDase (Cytosine Deaminase) activities, an antibody, a polypeptide inhibiting cellular division or transduction signals, a tumor suppressor gene (p53, Rb, p73 . . . ), a polypeptide activating host immune system, a tumor-associated antigen (MUC-1, BRCA-1, an HPV early or/and late antigen (E6, E7, L1, L2 . . . ) . . . ), optionally in combination with a cytokine gene.

The therapeutic gene may be engineered as a functional expression cassette, including a suitable promoter. The latter may be obtained from any viral, prokaryotic, e.g., bacterial, or eukaryotic gene (even from the gene of interest), be constitutive or regulable. Optionally, it may be modified in order to improve its transcriptional activity, delete negative sequences, modify its regulation, introduce appropriate restriction sites etc. Suitable promoters include but are not limited to adenoviral E1a, MLP, PGK (Phospho Glycero Kinase; Adra et al., Gene 60 (1987) 65-74; Hitzman et al., Science 219 (1983) 620-625), MT (metallothioneine; Mc Ivor et al., Mol. Cell Biol. 7 (1987), 838-848), α-1 antitrypsin, CFTR, surfactant, immunoglobulin, β-actin (Tabin et al., Mol. Cell Biol. 2 (1982), 426-436), SRα (Takebe et al., Mol. Cell. Biol. 8 (1988), 466-472), early SV40 (Simian Virus), RSV (Rous Sarcoma Virus) LTR, TK-HSV-1, SM22 (WO 97/38974), Desmin (WO 96/26284) and early CMV (Cytomegalovirus; Boshart et al., Cell 41 (1985) 521). Alternatively, promoters can be used which are active in tumor cells. Suitable examples include but are not limited to the promoters isolated from MUC-1 gene over expressed in breast and prostate cancers (Chen et al., J. Clin. Invest. 96 (1995), 2775-2782), CEA (Carcinoma Embryonic Antigen) over expressed in colon cancers (Schrewe et al., Mol. Cell. Biol. 10 (1990), 2738-2748), tyrosinase over expressed in melanomas (Vile et al., Cancer Res. 53 (1993), 3860-3864), ErbB-2 over expressed in breast and pancreas cancers (Harris et al., Gene Therapy 1 (1994), 170-175) and α-foetoprotein over expressed in liver cancers (Kanai et al., Cancer Res. 57 (1997), 461-465). The early CMV promoter is preferred in the context of the invention.

The expression cassette may further include additional functional elements, such as intron(s), secretion signal, nuclear localization signal, IRES, poly A transcription termination sequences, tripartite leader sequences and replication origins.

The vector in use in the present invention may comprise one or more gene(s) of interest. The different genes may be included in the same cassette or in different cassettes thus controlled by separate regulatory elements. The cassettes may be inserted into various sites within the vector in the same or opposite directions. According to another alternative, the different genes may be placed on different vectors.

Optionally, a therapeutic agent in use in the present invention can be associated with one or more stabilizing substance(s) such as lipids (i.e., cationic lipids such as those described in WO 98/44143, liposomes), nuclease inhibitors, polymers, chelating agents in order to prevent degradation within the human/animal body.

According to a preferred embodiment, the peptide of the present invention is operably coupled to the therapeutic agent. “Operably coupled” means that the components so described are in a relationship permitting them to function in their intended manner (i.e., the peptide promotes the targeting of the therapeutic agent to the desired cell). The coupling can be made by different means that are well known to those skilled in the art and include covalent, non covalent or genetic means.

Covalent attachment of peptides to the surface of the therapeutic agent may be performed through reactive functional groups at the surface of the therapeutic agent, optionally with the intermediary use of a cross linker or other activating agent (see for example Bioconjugate techniques 1996; ed G Hermanson; Academic Press). The functional groups of the therapeutic agent may be modified to be reactive towards specific amino acid groups of the peptide. In particular, coupling may be done with (i) homobifunctional or (ii) heterobifunctional crosslinking reagents, with (iii) carbodiimides, (iv) by reductive amination or (vi) by activation of carboxylates.

Homobifunctional cross linkers including glutaraldehyde and bis-imidoester like DMS (dimethyl suberimidate) can be used to couple amine groups of peptides to lipoplexes containing diacyl amines such as phosphatidylethanolamine (PE) residues. Other examples are given in Bioconjugate techniques (1996) 188-228; ed G Hermanson; Academic Press).

Many heterobifunctional cross linkers can be used in the present invention, in particular those having both amine reactive and sulfhydryl-reactive groups, carbonyl-reactive and sulfhydryl-reactive groups and sulfhydryl-reactive groups and photoreactive linkers. Suitable heterobifunctional crosslinkers are described in Bioconjugate techniques (1996) 229-285; ed G Hermanson; Academic Press) and WO 99/40214. Examples of the first category include but are not limited to SPDP (N-succinimidyl 3-(2-pyridyldithio)propionate), SMBP (succinimidyl-4-(p-maleimidophenyl)butyrate), SMPT (succinimidyloxycarbonyl-α-methyl-(α-2-pyridyldithio)toluene), MBS (m-maleimidobenzoyl-N-hydroxysuccinimide ester), SIAB (N-succinimidyl(4 iodoacetyl)aminobenzoate), GMBS (γ-maleimidobutyryloxy)succinimide ester), SIAX (succinimidyl-6-iodoacetyl amino hexonate, SIAC (succinimidyl-4-iodoacetyl amino methyl), NPIA (p-nitrophenyl iodoacetate). The second category is useful to couple carbohydrate-containing molecules (e.g., env glycoproteins, antibodies) to sulfydryl-reactive groups. Examples include MPBH (4-(4-N maleimidophenyl)butyric acid hydrazide) and PDPH (4-(N-maleimidomethyl)cyclohexane-1-carboxyl-hydrazide (M₂C₂H and 3-2(2-pyridyldithio)proprionyl hydrazide). As an example of the third category, one may cite ASIB (1-(p azidosalicylamido)-4-(iodoacetamido)butyrate). Another alternative includes the thiol reactive reagents described in Frisch et al., (Bioconjugate Chem. 7 (1996) 180-186).

Coupling (iii) involves, e.g., amine groups of underivatized PE present in lipoplexes that can participate in the carbodiimide reaction with carboxylate groups on proteins.

Coupling (iv) may be performed, e.g., via imine formation followed by reduction using a cyanoborohydrate.

Coupling (vi) may involve, e.g., an NHS ester derivative of lipoplexe and a peptide amine group to produce stable amide bond linkages.

Another example uses a maleimide-sulfhydryl bond involving a sulfhydryl group and a sulfhydryl reactive group. For example SATA (N-succinimidyl S-acelythioacetate) can be used to introduce a sulfhydryl group whereas sulfo SMCC (sulfosuccinimidyl 4-(N-maleimidomethyl)cyclo hexane 1-carboxylate) can be used to introduce a maleimide group resulting in a covalent thioether bond.

Another preferred linker is a polymer such as polyethylene glycol (PEG) or its derivatives. Preferably, such a polymer has an average molecular weight comprised between 200 to 20000 Da. For example, tresyl-MPEG can be used to couple an, amino group present on Lys residues (see for example WO 99/40214). Other means to conjugate two partners via PEG are described in the literature (in Bioconjugate techniques (1996) 606-618; ed G Hermanson; Academic Press and Frisch et al., Bioconjugate Chem. 7 (1996) 180-186).

Non covalent coupling includes electrostatic interactions, for example between a cationic peptide and a negatively charged plasmidic or viral vector or between an anionic peptide and a cationic synthetic vector. Another alternative consists in using affinity components such as Protein A, biotin/avidin, antibodies, which are able to associate non covalently or by affinity on the one hand the peptide of the invention and on the other hand the therapeutic agent. Concerning lipoplexes, biotinylated PE derivatives can be used to interact non covalently with avidin peptide conjugates or with other biotinylated peptides using avidin as a bridging molecule (Bioconjugate techniques (1996) 570-591; ed G Hermanson; Academic Press). Coupling with viral vectors may use biotinylated antibodies directed against a capsid epitope and streptavidin-labeled antibodies directed against a peptide of the invention (Roux et al., Proc. Natl. Acad Sci USA 86 (1989) 9079).

Covalent coupling with plasmidic vectors may use an alkylating agent (Sebestyen et al., Nat. Biotechnol. 16 (1998) 80-85; Ciolina et al., Bioconjug. Chem. 10 (1999) 49-55; Zanta et al., Proc. Natl. Acad. Sci. USA 96 (1999) 91-96). Non covalent coupling may be achieved by using PNA (Peptide Nucleic Acid) or triple helix (Neves et al., Cell Biol. Toxicol. 15 (1999) 193-202; Neves et al., FEBS Lett. 453 (1999) 41-45) or by any coupling agent interacting with nucleic acids, such as anti DNA immunoglobulins as described in WO 97/02840 or polycationic compounds such as polylysine. Bifunctional antibodies directed against each of the coupling partners are also suitable for this purpose.

Genetic coupling is more particularly intended for coupling a peptide according to the invention and a viral vector. Advantageously, a nucleic acid encoding such a peptide can be genetically inserted in addition to or in place of a native viral sequence that encodes a polypeptide exposed at the viral surface, to make the peptide of the invention expressed at the surface of the virus particle. Insertion sites can be selected on the basis of three-dimensional data in order to identify regions that are non essential for virus integrity. Peptide insertion can be made at any location, at the N-terminus, the C-terminus or between two amino acid residues of the viral polypeptide. Preferably the insertion of the peptide is made in frame and does not disrupt the viral open reading frame.

Suitable surface-exposed polypeptides include the external proteins of a poxviral vector (e.g., the expression products of the A27L (p14 protein), L1R, A14L, A17L (p21 protein), D8L, A9L, E10R and H3L genes of an IMV particle or the expression products of the B5R, A34R and HA genes of an EEV particle as described in EP-1,146,125), the envelope protein of a retroviral vector and an adenoviral capsid protein. Said adenoviral capsid protein is preferably selected among the group consisting of fiber, hexon, penton base and pIX proteins. In the context of the present invention, the peptide of the invention is inserted into an adenoviral fiber (Ad2 fiber-encoding gene is described in Herissé et al; Nucleic Acid Res. 9 (1981) 4023-4042; Ad5 fiber-encoding gene is described in Chroboczek et al., Virol. 161 (1987) 549-554). Preferably, the fiber into which the peptide of the invention is genetically inserted is modified. In this respect, the fiber sequences that ensure proper trimerization and association with the penton base complex are preserved whereas those coding for the CAR binding-site (Roelvink et al., Science 286 (1999) 1568-1571) are altered. Insertion in different loops of the knob domain, more specifically in AB, CD, DG, GLUCOSYLATED HYDROXYSTILBENE, HI and IJ loops, or at the C-terminus (i.e., just upstream to the STOP codon or in addition to or in place of the few residues preceding the STOP codon) can be envisaged. Examples of appropriate locations are illustrated in WO 94/10323, WO 95/26412, WO 95/05201, WO 96/26281, WO 98/44121 and FR-99/10859. Insertion of the peptide sequence within the HI loop of the knob of an adenoviral fiber is preferred (e.g., between residues 545 and 546 of the Ad5 fiber).

In the context of the present invention, it is preferred that the fiber protein into which the peptide is inserted be further modified, e.g., in order to reduce or abolish the interaction with at least one cellular receptor that normally facilitates directly or indirectly virus binding to cells. Such receptors include but are not limited to the coxsackievirus-adenovirus receptor (termed CAR) (Bergelson et al., Science 275 (1997), 1320-1323; Tomko et al., Proc. Natl. Acad. Sci. USA 94 (1997), 3352-3356), the alpha 2 domain of the major histocompatibility complex class I molecule (Hong et al., EMBO J. 16 (1997), 2294-2306), the cell-surface heparan sulfate glycosaminoglycans (HSG) (Dechecchi et al., Virology 268 (2000), 382-390; Dechecchi et al., J. Virol. 75 (2001), 8772-8780), and cell-surface sialic acid. Preferably, the modified fiber in use in the present invention contains one or more mutation(s) (e.g., substitution, deletion and/or addition of one or more residue), aimed to reduce or abolish the interaction with at least one cellular receptor which normally facilitates virus binding to a cell, without disturbing the structure of the fiber (i.e., trimerization and/or association with penton base). Preferably, the modified adenoviral fiber is capable of trimerizing when produced in an eukaryotic host cell. Point mutation that abolish interaction with CAR are preferred. In this respect, a number of CAR binding ablated fibers have been described in the literature, including but not limited to those describes in Bewley et al., (Science 286 (1999), 1579-1583), Roelvink et al., (Science 286 (1999), 1568-1571), Kirby et al., (J Virol 73 (1999), 9508-9514), Kirby et al., (J. Virol. 74 (2000), 2804-2813), Leissner et al., (Gene Ther. 8 (2001), 49-57), Jakubczak et al., (J. Virol. 75 (2001), 2972-2981), WO 98/44121, WO 01/16344 and WO 01/38361). A modified Ad5 fiber comprising the substitution of the serine residue in position 408 by a glutamic acid (S408E) is absolute preference in this respect. The modified adenoviral fiber can also be modified to reduce or abolish interaction with other cellular receptors (e.g., HGS and/or sialic acid-containing receptor), optionally in combination with one or more CAR-ablating mutation(s). Mutations reducing or abolishing interaction with HGS and/or sialic acid-containing receptor have been described in European Application EP-O-2,360,204.8. A suitable HGS-ablated modified fiber includes, but is not limited to, an Ad5 fiber comprising the substitution of the lysine in position 506 by a glutamine and the substitution of the histidine in position 508 by a lysine.

Introduction of the peptide encoding nucleic acid in an adenoviral gene encoding the penton base or hexon may be performed as described in WO 96/07734 and U.S. Pat. No. 5,559,099. Where the peptide is inserted or replace a portion of the penton base, preferably it is within the hypervariable regions to ensure presentation at the viral surface. Where the peptide is inserted or replace a portion of the hexon, preferably it is within the hypervariable regions. A suitable example is an adenovirus hexon comprising a deletion of about 13 amino acid residues from the HVR5 loop, corresponding to about amino acid residue 269 to about amino acid residue 281 of the Ad5 hexon and insertion of the peptide at the site of the deletion, eventually connected by a first spacer at the N-terminus and a second spacer at the C-terminus of the peptide.

Also, the peptide can be genetically inserted in an adenoviral pIX protein, at any location but with a special preference for insertion at the C-terminus or within the C-terminal portion of pIX (e.g., in replacement of or in addition to one or more residues located within the 40 pIX residues preceeding the STOP codon). Where the peptide is inserted in the pIX protein, preferably pIX is also mutated in the coil coiled domain (as described for example in Rosa-Calavatra et al., 2001, J. Virol. 75, 7131-7141). A suitable mutated pIX includes, but is not limited to, the Ad5 pIX comprising the substitution of the leucine in position 114 by a proline and the substitution of the valine in position 117 by an aspartic acid.

The present invention also encompasses the use of peptide spacer (or linker) to further improve presentation of the peptide of the present invention at the viral surface. The term [symbol] peptide spacer [symbol] or [symbol] linker [symbol] as used herein refers to a sequence of about one to 20 amino acids that is included to connect the peptide to the polypeptide exposed at the viral surface. The spacer is preferably made up of 7 to 30 amino acid residues with high degrees of freedom of rotation. Preferred amino acids for the spacer are alanine, glycine, proline and/or serine. In specific embodiments, the spacer is a peptide having the sequence Ser-Ala, Pro-Ser-Ala or Pro-Gly-Ser or a repetition thereof.

A preferred composition of the invention comprises a peptide having the sequence X₁HEWSYLAPYPWFX₂ (SEQ ID NO: 74), wherein X₁ and X₂ are as defined above.

Alternatively, coupling between the peptide of the invention and the therapeutic agent may be done in the organism at the site of the cells to be targeted. According to such an embodiment, non covalent coupling is preferred. For example, one may envisage to introduce in the organism or to the target cell (i) the peptide according to the invention associated with a first affinity component (e.g., biotin) and (ii) the therapeutic agent associated with a second affinity component capable to bind the first one (e.g., avidin). Preferably, (i) is introduced before (ii).

As indicated before, the composition of the present invention may comprise a nucleic acid encoding the peptide of the invention instead of the peptide as such. According to a first alternative, the nucleic acid encoding such a peptide can be fused to a therapeutic gene. The fusion sequence can be placed under the control of suitable elements allowing its expression (e.g., a promoter) and incorporated in a conventional vector which can be introduced into an organism to be treated in order to locally express a fusion polypeptide that combines both targeting and therapeutic properties. A preferred fusion sequence is obtained by fusing the nucleic acid encoding a tumor-targeting peptide of the present invention and an immunostimulatory gene (e.g., B7.1) and is engineered to include functional elements allowing secretion of the fusion polypeptide outside the expressing cells (presence of a signal sequence). Injection of such a fusion sequence to an organism having cancer will result in the synthesis and secretion of a fusion polypeptide allowing the targeting of the tumor cells present in the organism and the in situ delivery of the immunostimulatory polypeptide capable of enhancing the anti-tumoral response. Another alternative would be to incorporate into two adenoviral particles on the one hand genes encoding retroviral helper functions (gag/pol and env genes) with the env gene comprising a nucleic acid encoding a peptide according to the invention and on the other hand a conventional retroviral vector engineered to express a therapeutic gene. Cells co-infected with the two adenoviral particles will produce infectious retroviral particles with an envelope exposing the targeting peptide. The use of a tumor-targeting peptide will allow local targeting of tumoral cells.

In accordance with the goal pursued by the present invention, the peptide and/or the therapeutic agent may be modified to improve or stabilize the coupling. In particular, the peptide may be extended by a spacer at the N or C-terminus to facilitate its accessibility to target cells after coupling.

Moreover, a composition according to the invention may comprise one or more peptides of the invention that may or may not be fused (i.e., in tandem). For example, when it is desirable to enhance the specificity of the composition of the invention towards a specific target, it may be advantageous to use a combination of targeting peptides.

A composition according to the invention may be manufactured in a conventional manner for local, systemic, oral, rectal or topical administration. Suitable routes of administration include but are not limited to intragastric, subcutaneous, intradermal, aerosol, instillation, inhalation, intracardiac, intramuscular, intravenous, intraarterial, intraperitoneal, intratumoral, intranasal, intrapulmonary or intratracheal routes. The administration may take place in a single dose or a dose repeated one or several times after a certain time interval. The appropriate administration route and dosage vary in accordance with various parameters, for example, with the individual, the disorder to be treated, the therapeutic agent or with the gene of interest to be transferred. As far as viral vectors are concerned, the corresponding viral particles may be formulated in the form of doses of between 10⁴ and 10¹⁴ iu (infectious unit), advantageously between 10⁵ and 10¹³ iu and preferably between 10⁶ and 10¹² iu. The titer may be determined by conventional techniques (see for example Lusky et al., 1998, supra). Doses based on a plasmid or synthetic vector may comprise between 0.01 and 100 mg of DNA, advantageously between 0.05 and 10 mg and preferably between 0.5 and 5 mg. The formulation may also include a pharmaceutically acceptable diluent, adjuvant, carrier or excipient. In addition, a composition according to the present invention may include buffering solutions, stabilizing agents or preservatives adapted to the administration route. For example, an injectable solution may be liquid or in the form of a dry powder (lyophylized . . . etc) that can be reconstituted before use. Compositions for topical administration may be in the form of creams, ointments, lotions, solutions or gels. Compositions for intra-pulmonary administration may be in the form of powder, spray or aerosol.

A composition according to the present invention can be administered directly in vivo by any conventional and physiologically acceptable administration route, for example by intraarterial injection, into an accessible tumor, into the lungs by means of an aerosol or instillation, into the vascular system using an appropriate catheter, as well as by intradermal, subcutaneous, intramuscular, systemic (e.g., intravenous) routes etc. The ex vivo approach may also be adopted which consists in removing cells from the patient (bone marrow cells, peripheral blood lymphocytes, myoblasts and the like . . . ), introducing the composition of the invention in accordance with the techniques of the art and readministering them to the patient. As mentioned above, administration may be performed according to a two steps procedure, the first step consisting of administering a peptide of the invention associated with a first affinity component in order to target the desired cells and the second step consisting of administering the therapeutic agent associated with a second affinity component capable of binding the first one.

As described above, the present invention also encompasses vectors or particles that have been modified to allow preferential targeting of a particular target cell. A characteristic feature of targeted vectors/particles of the invention (of both viral and non-viral origins, such as polymer- and lipid-complexed vectors) is the presence at their surface of a peptide according to the invention, e.g., in order to have the peptide capable of recognizing and binding to a cellular and surface-exposed component. Therefore, the present invention also provides an adenoviral vector comprising a peptide of the invention as defined above, so that said peptide is exposed at the surface of the viral particle. Said adenoviral vector is as defined above and encompasses adenoviral genome (nacked DNA), plasmid comprising such a genome, adenoviral particles or empty adenoviral capsids. Advantageously, said peptide is genetically inserted in addition to or in place of one or more residue(s) of a native capsid adenoviral protein. The capsid adenoviral protein is selected among the group consisting of fiber, hexon, penton-base and pIX proteins.

According to a first alternative, the adenoviral capsid protein is a fiber protein and the peptide of the present invention is genetically inserted in addition to or in place of one or more residues of the fiber, with a special preference for insertion within the HI loop or at the C-terminus of said fiber protein. It is within the scope of the present invention that the fiber protein into which is inserted the peptide of the invention can be further modified. A preferred embodiment relates to an adenoviral vector having a modified fiber containing one or more mutation(s) aimed to reduce or abolish the interaction of said fiber with at least one cellular receptor which normally facilitates virus binding to a cell. Preferably, the modified fiber contains one or more mutation(s) aimed to reduce or abolish the interaction of said fiber with at least the coxsackievirus and adenovirus receptor (CAR), as described above, with a special preference for an Ad5 fiber comprising the substitution of the serine residue in position 408 by a glutamic acid. Alternatively or in combination, the fiber can be modified to reduce or abolish the interaction with the HGS and/or the sialic acid-containing receptors.

According to a second alternative, the adenoviral capsid protein is a pIX protein and the peptide of the present invention is genetically inserted at the C-terminus or within the C-terminal portion of said pIX protein. Preferably, the pIX protein into which is inserted the peptide of the invention can be further modified. In this context, the modified pIX can contain one or more mutation(s), with a special preference with one or more mutation(s) in its coil-coiled domain (as described for example in Rosa-Calavatra et al., 2001, J. Virol. 75, 7131-7141). In the context of the present invention, the fiber protein of the adenoviral vector having a peptide of the invention inserted in the pIX protein can be either native or modified as described above (e.g., CAR-ablated fiber).

A preferred adenoviral vector according to the present invention comprises a peptide having the sequence X₁ HEWSYLAPYPWFX₂ (SEQ ID NO: 74), wherein each X₁ and X₂ independently of one another represents any amino acid sequence of n amino acids, n varying from 0 to 50 and n being identical or different in X₁ and X₂. An even more preferred adenoviral vector according to the invention is an Ad5 adenoviral vector, having the peptide HEWSYLAPYPWF genetically inserted within the HI loop of the adenoviral fiber protein, and wherein said fiber comprises the substitution of the serine residue in position 408 by a glutamic acid; with a special preference for insertion of the peptide HEWSYLAPYPWF between residues 545 and 546 of said adenoviral fiber protein.

As mentioned above, the adenoviral vector according to the invention can be replication-defective and/or recombinant.

The adenoviral vector of the present invention may comprise one or more peptide according to the invention. They may be inserted in the same (e.g., an adenoviral fiber or pIX) or different viral capsid proteins (e.g., an adenoviral fiber and pIX), and in the same location (e.g., in tandem) or at different locations (within the HI loop and at the C-terminus of the fiber).

The present invention also encompasses host cells infected with an adenoviral vector according to the invention. For the purpose of the invention, the term “host cells” should be understood broadly without any limitation concerning particular organization in tissue, organ, etc or isolated cells of a mammalian (preferably a human). Such cells may be unique type of cells or a group of different types of cells and encompass cultured cell lines, primary cells and proliferative cells from mammalian origin, with a special preference for human origin. Suitable host cells include but are not limited to hematopoïetic cells (totipotent, stem cells, leukocytes, lymphocytes, monocytes, macrophages, APC, dendritic cells, non-human cells and the like), pulmonary cells, tracheal cells, hepatic cells, epithelial cells, endothelial cells, muscle cells (e.g., skeletal muscle, cardiac muscle or smooth muscle), fibroblasts . . . etc. Moreover, according to a specific embodiment, the eukaryotic host cell of the invention can be further encapsulated. Cell encapsulation technology has been previously described (Tresco et al., ASAIO J. 38 (1992), 17-23; Aebischer et al., Human Gene Ther. 7 (1996), 851-860).

The present invention also relates to a composition comprising an adenoviral vector according to the invention and a pharmaceutically acceptable carrier. The characteristic features of such a composition is as described above.

Finally, the present invention also provides for the use of a composition according to the invention, for the preparation of a drug intended for gene transfer and preferably for the treatment of human or animal body by gene therapy. Within the meaning of the present invention, gene therapy has to be understood as a method for introducing any therapeutic gene into a cell. Thus, it also includes immunotherapy that relates to the introduction of a potentially antigenic epitope into a cell to induce an immune response which can be cellular or humoral or both. The use of a composition according to the invention is dependent upon the targeting properties of the peptide included in said composition. A composition comprising a heart targeting peptide is preferably used for the treatment or prevention of any disease affecting the heart or its vasculature, such as coronary heart diseases, heart failure, heart hypertrophy, infarction, myocarditis, ischemia, restenosis, atherosclerosis, muscular and the like. A preferred use for a composition comprising a tumor targeting peptide consists in treating or preventing cancers, tumors and diseases which result from unwanted cell proliferation. One may cite more particularly cancers of breast, uterus (in particular, those induced by a papilloma virus), prostate, lung, bladder, liver, colorectal, pancreas, stomach, esophagus, larynx, central nervous system, blood (lymphomas, leukemia, etc.), melanomas and mastocytoma.

The present invention also encompasses the use of the adenoviral vector according to the present invention, for the preparation of a drug intended for gene transfer. A preferred use is for targeting a tumor cell, with a special preference for a colon tumor cell or a breast tumor cell, especially when referring to the embodiment according to which the adenoviral vector comprises a peptide having the sequence X₁HEWSYLAPYPWFX₂ (SEQ ID NO: 74).

The present invention also relates to a method of treatment in which a therapeutically effective amount of a peptide or a composition according to the invention is administered to a patient in need of such a treatment. [symbol] Treatment [symbol] as used herein refers to prophylaxis and therapy. A [symbol] therapeutically effective amount of a peptide or a composition [symbol] is a dose sufficient to the alleviation of one or more symptoms normally associated with the disease desired to be treated. A method according to the invention is more intended for the treatment of the diseases listed above.

The present invention also provides a method for the treatment or prevention of a cancer or tumor, comprising administering a therapeutically effective amount of an adenoviral vector according to the invention to a patient in need of such treatment. Such a cancer or tumor is preferably a breast or a colon cancer or tumor, especially when the adenoviral vector comprises a peptide having the sequence X₁HEWSYLAPYPWFX₂ (SEQ ID NO: 74).

The disclosure of all patents, publications including published patent applications, and database entries cited in the present application are hereby incorporated by reference in their entirety to the same extend as if each such individual patent, publication and database entry were specifically and individually indicated to be incorporated by reference and were set forth in its entirety herein.

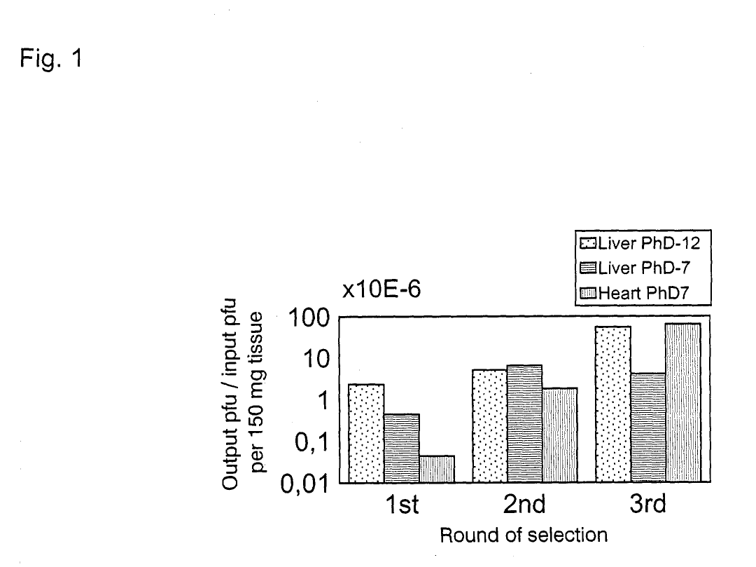

FIG. 1 represents schematically the total number of recovered phages (output pfu (plaque forming units)) calculated per 150 mg organ (liver or heart), for three rounds of in vivo selection with different phage display libraries. All numbers are divided with the titers obtained from the injected inputs to be able to compare between mice.

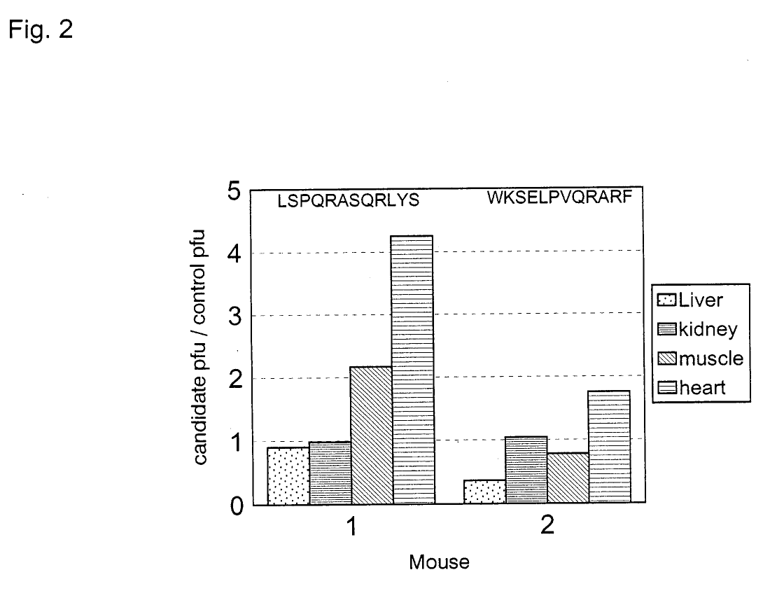

FIG. 2 represents schematically an example of in vivo testing of specificity of candidate phages with a co-injected negative control. The sequence of the displayed peptides is shown in the figure.

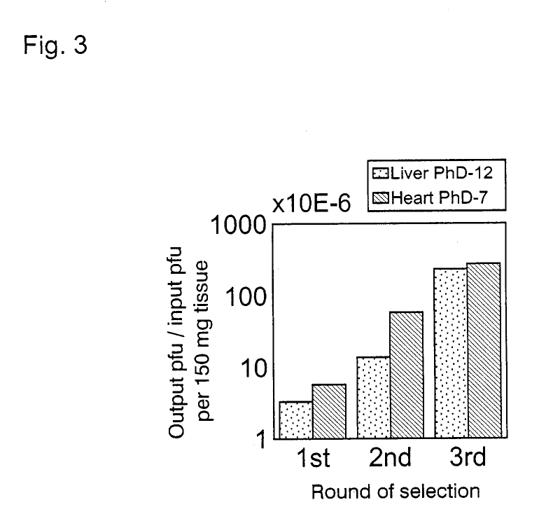

FIG. 3 represents schematically the total number of recovered phages (output pfu) calculated per 150 mg of fixed and minced organ (liver or heart), for three rounds of ex vivo selection with two different phage display libraries. All numbers are divided by the titers obtained from the input amount of phages to be able to compare between mice.

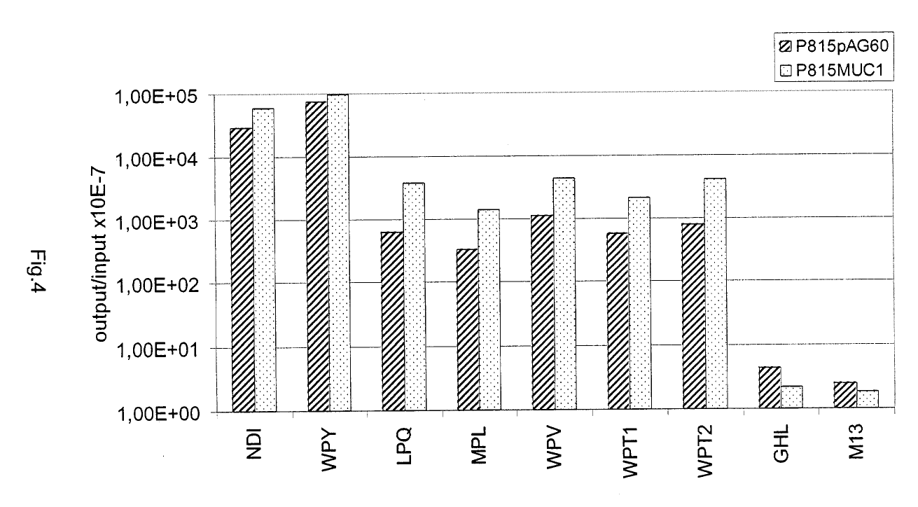

FIG. 4 represents schematically an example of in vitro testing of the specificity of candidate phages binding to P815 cells. The sequence of the displayed peptides is indicated by the three amino acid motif present at the N-terminus. M13 phage and a non-selected phage (GHL) are used as negative controls.

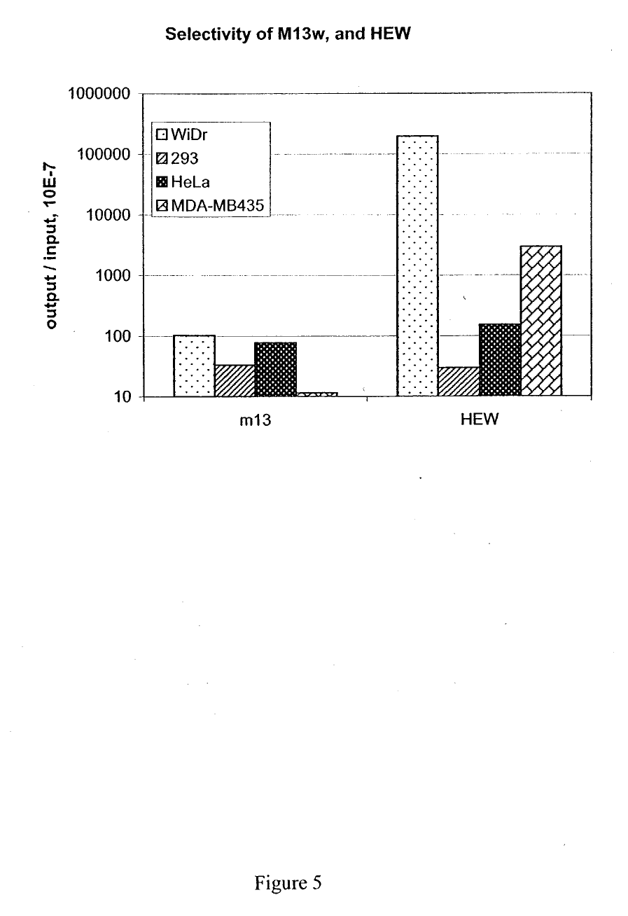

FIG. 5 represents schematically an example of in vitro testing of the specificity of candidate phages binding to WiDr cells in comparison to other cells. The sequence of the displayed peptides is indicated by the three amino acid motif present at the N-terminus. M13 phage and three non-specific phages (not shown) are used as negative controls.

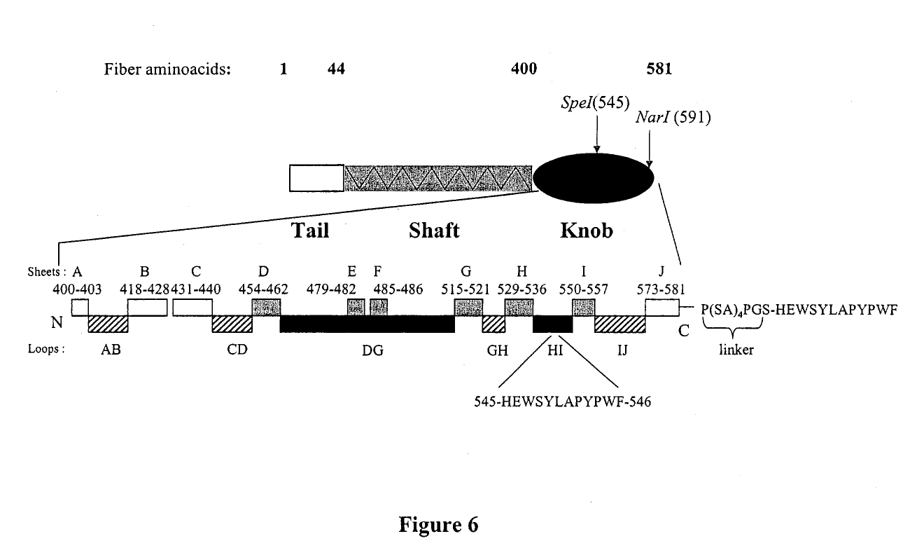

FIG. 6 provides a schematic representation of a modified adenoviral fiber comprising the HEW tumor specific peptide inserted into the knob domain of the Ad5 wild-type or S408E fiber, either at the carboxy-terminus after a short flexible linker, or in the HI loop.

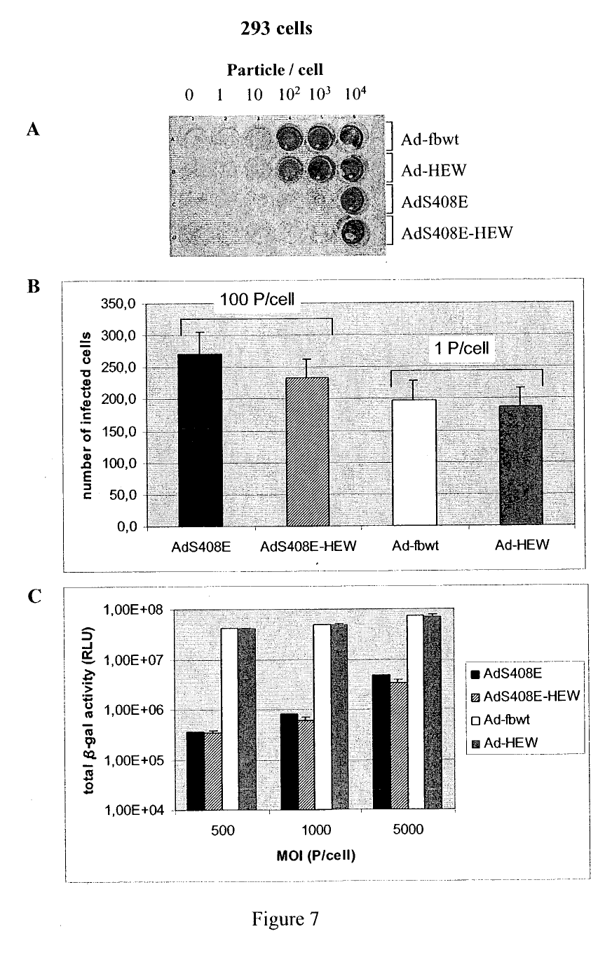

FIG. 7 illustrates the efficiency of infection of HEW viruses in 293 control cells. 2×10⁵ 293 cells in monolayers were infected with the different indicated viruses at 1 to 10⁴ P (particle)/cell. At 20 hours post-infection, cells were fixed and stained for β-galactosidase expression (A). The number of infected cells was quantified by counting of blue cells (B). Alternatively, cells infected with increasing viral titers were lysed and β-galactosidase activity of the supernatant was monitored using a chemiluminescent detection kit (C).

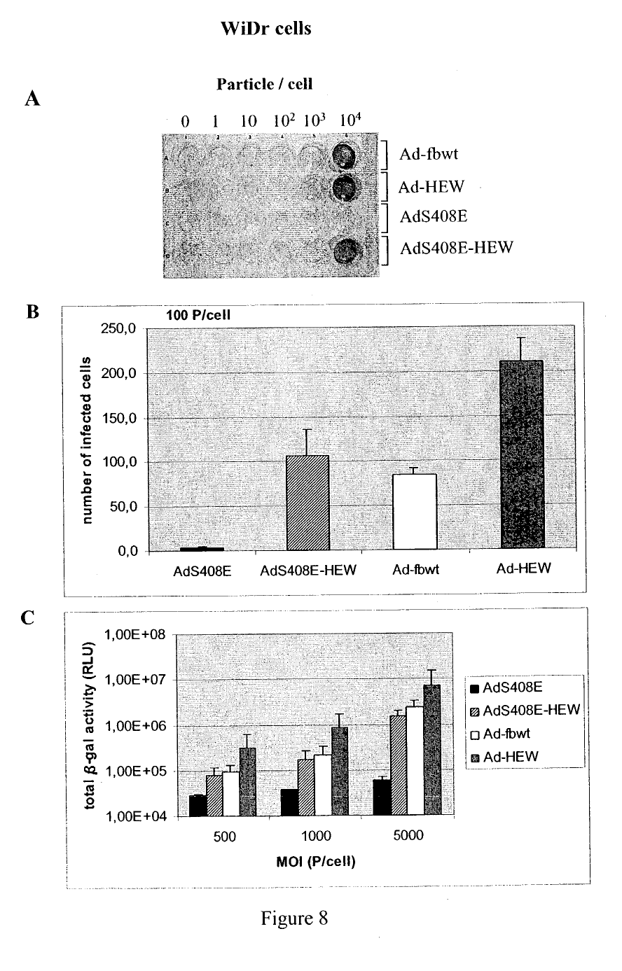

FIG. 8 illustrates the efficiency of infection of HEW viruses in WiDr cells. 2×10⁵ WiDr cells in monolayers were infected with the different indicated viruses at 1 to 10⁴ P (particle)/cell. At 20 hours post-infection, cells were fixed and stained for β-galactosidase expression (A). The number of infected cells was quantified by counting of blue cells (B). Alternatively, cells infected with increasing viral titers were lysed and β-galactosidase activity of the supernatant was monitored using a chemiluminescent detection kit (C).

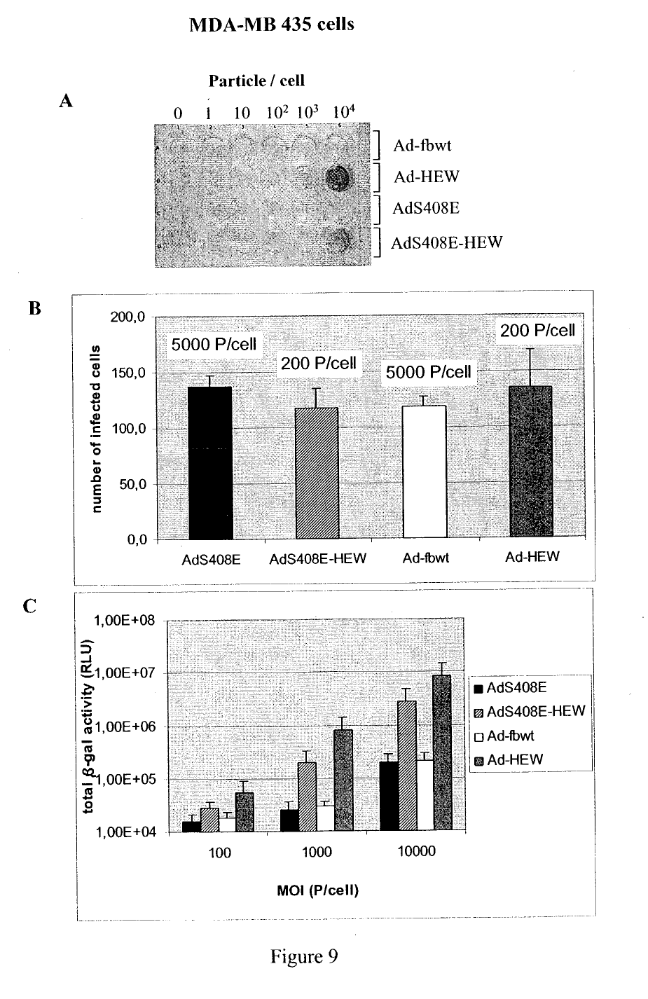

FIG. 9 illustrates the efficiency of infection of HEW viruses in MDA-MB435 cells. 2×10⁵ MDA-MB435 cells in monolayers were infected with the different indicated viruses at 1 to 10⁴ P (particle)/cell. At 20 hours post-infection, cells were fixed and stained for β-galactosidase expression (A). The number of infected cells was quantified by counting of blue cells (B). Alternatively, cells infected with increasing viral titers were lysed and β-galactosidase activity of the supernatant was monitored using a chemiluminescent detection kit (C).

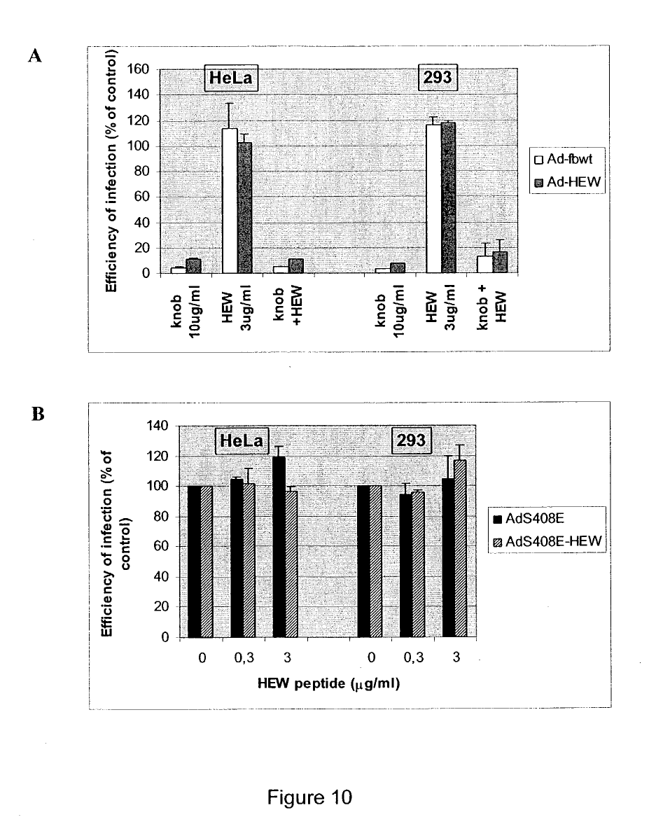

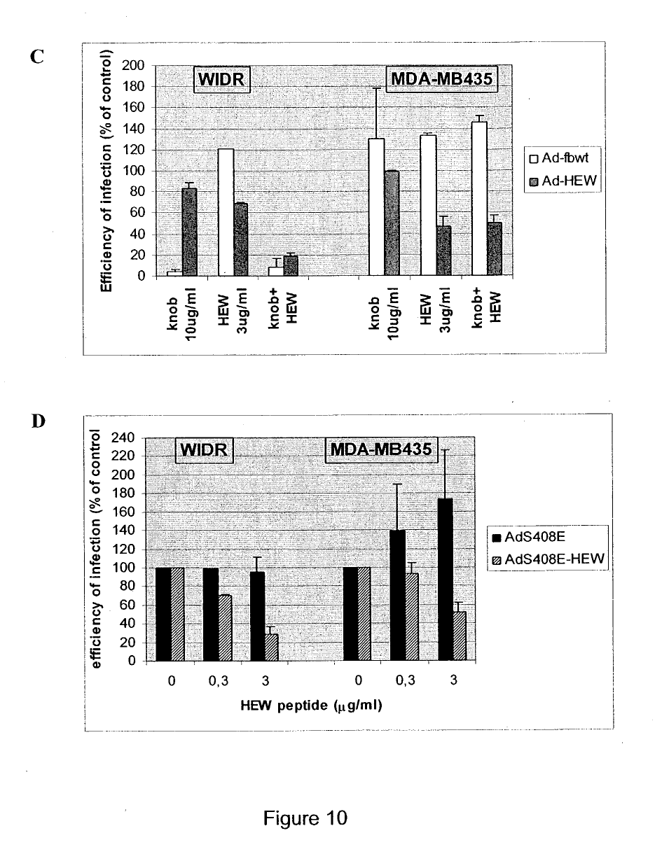

FIG. 10 illustrates the entry pathway of control and HEW-containing viruses in control and target cells. Soluble knob peptide (10 μg/ml), or HEW-K16 peptide (0.3 or 3 μg/ml), or a mix of both were incubated with control (293, HeLa) and target (WiDr, MDA-MB435) cells for 30 minutes at 37° C. The different viruses were then added to the cells at a fixed MOI. At 20 hours post-infection, cells were lysed and total β-galactosidase activity was monitored. The efficiency of infection was expressed as the percentage of β-galactosidase activity in the absence of competitor (% of control).

The following examples serve to illustrate the present invention.

Peptides of the invention have been identified using a phage display peptide library. This technology conventional in the domain of the art is detained in the following documents (Scott et al., Science 249 (1990) 368; Cwirla et al., Proc. Natl. Acad. Sci. USA 87 (1990) 6378; Devlin et al., Science 249 (1990) 404; Romanczuk et al., Hum. Gene Ther. 10 (1999) 2615; Samoylova et al., Muscle and Nerve 22 (1999) 460). One of the most commonly used phages for phage display libraries is the filamentous phage M13. The M13 phage can be designed to display on its surface a foreign peptide fused to a coat protein and to harbor the gene for the fusion protein within its genome. The pIII and pVIII surface proteins of the M13 virion are currently used in phage display. The pill protein is present in 3 to 5 copies closely positioned to each other. The pVIII protein is present in about 2700 copies distributed over the surface of the phage. Random peptide sequences can be incorporated at the N-terminus of either proteins.

Different strategies are available to select phages that selectively target a desired cell type. The first screening method involves in vivo injection of the phage display library, isolation of the target tissue and amplification by subjecting the retained phages to two or more rounds of in vivo selection towards the same organ (Rajotte et al., J. Clin. Invest. 102 (1998) 430-437; Pasqualini et al., Nature 380 (1996) 364-366; Pasqualini et al., Nature Biotechnology 15 (1997) 542-546). The in vivo approach is applicable for targeting various tissues (injection in wild type animals), tumors (using tumor animal models) and affected cells (injection in various animal models, for example artherosclerotic plaques in KO mice or ischaemic limbs).

In the second approach (ex vivo), organs or tissues are isolated, cut in small pieces, slightly fixed and then incubated with the phage library. Unbound phages are removed by washing, and bound phages are eluted at low pH or by directly adding host bacteria. The retained phages are amplified in bacteria and then further enriched by reexposure to the target (Van Ewijk et al., Proc. Natl. Acad. Sci. USA 94 (1997) 3903; Odermatt et al., J. Am. Soc. Nephr. 10 (1999) 448). The ex vivo selection scheme allows the use of human samples as the selecting tissues, for example biopsies from heart muscle, tumors or artherosclerotic plaques.

Finally, the in vitro selection strategy is based on the adsorption of phages to cultured cells (Waters et al., Immunotechnology 3 (1997) 21; Barry et al., Nature Medecine 2 (1996) 299; Samoylava et al., Muscle and Nerve 22 (1999) 460). A pre-adsorption step can be realized to eliminate the phages that exhibit a strong unspecific binding, for example those which display long stretches of positively and negatively charged amino acids. For this purpose, the library may be pre-adsorbed to unrelated cell lines (different from the target cells), non transformed cells from the same tissue or plastic surfaces. Then, the phage display library is incubated with the target cells grown in culture. Unbound phages are washed away and bound phages are eluted, recovered and amplified. When tumor targeting is concerned, the in vitro selection is performed on cultured tumor cell lines from various origins or primary tumor cells prepared from tumor tissues. Furthermore, the extracellular matrix (ECM) of tumors represents another potential target. ECM can be isolated from tumors (i.e., matrigel), fixed to tissue cuture dishes and used to select phages. Also, phages can be selected against isolated molecules (Burg et al., Cancer Res. 59 (1999) 2869; Koivuen et al., Nature Biotechn. 17 (1999) 768).

The in vitro selection on cell lines can be extended to select peptides that are specific for certain cell-surface exposed proteins. Several tumor specific cell surface antigens are known and could be used as specific addresses. Some examples are listed in Table 1. In this approach, the cell-surface receptor is expressed in a cell line after stable transformation of appropriate expression plasmids. Phages are first pre-selected against the parental cell line which does not express the receptor and then positively selected on the receptor expressing cells. This allows to select peptides against target proteins which are not available in purified form and has the additional advantage of displaying a receptor in the context of the cell membrane.

TABLE 1

| TUMOR ASSOCIATED | | |
|---|---|---|
| ANTIGEN | TUMOR | REFERENCE |
| MUC-1 | Breast, pancreas, | Croce et al. Anticancer |
| | ovarian, cancer | Res. 17 (1997), 4287-92 |
| HER2/neu | Breast, ovarian | Kirpotin at al. Biochem. |
| (ERBB2)receptor | endometrial, lung, | 36 (1997) 66-75 |
| | gastric, bladder, | |
| | prostate | |
| CEA (carcinoembryonic | Colon, lung | Jessup et al. Semin. Surg |
| antigen) | | Oncol 15 (1998) 131-140 |
| Folate receptor | Ovarian | Gottschalk et al. Gene |
| | | Therapy 1 (1994) 185-91 |
| EGF receptor | Lung | Christiano et al. Cancer |
| | | Gene Ther 3 (1996) 4-10 |
| Melanocortin receptor 1 | Melanoma | Szardenings et al. J Biol |
| | | Chem 272 (1997) 27943-8 |
| Integrin alpha v beta 3 | Tumor vessels | Varner et al. Curr Opin |
| | | Cell Biol 8 (1996) 724-30 |

At the end of any of the above described selection procedures, a limited number of retained phages are individually isolated, amplified and subsequently characterized by determining the sequence of the DNA insert encoding the display peptide. In addition, the amino acid sequences will be aligned to identify motifs that are unique for a given cell. The most abundant sequences are then tested for specific binding.

To confirm the binding specificity of a selected phage in vivo, it will be injected into mice and different organs will be recovered. The accumulation of phages in a given organ will be followed by determining the number of phages recovered from various organs or by performing quantitative PCR for phage specific genomic sequences. The ratio of target/non target organ for the phage or DNA recovery represents a measurement for its specificity. In the literature, ratios of 2 to 35 have been described (Arap et al. Science 279 (1998) 377; Pasqualini et al. Nat. Biotech. 15 (1997) 542; U.S. Pat. No. 5,622,699). This ratio will be compared with the one obtained with unselected phage pools, unselected individual phages or wild type phages. Phage accumulation can also be followed by immunohistochemistry using anti-M13 antibodies. This aspect is particularly relevant to identify more precisely the target tissue (vasculature, tumor cell, ECM). Furthermore, selected phages could be injected in the presence of the free peptide or a GST (gluthation S transferase) fusion peptide to demonstrate specific targeting in a competition assay. In addition, specificity can be tested by linking a tumor-targeting peptide to a chemotherapeutic drug (i.e., doxorubicin) and demonstrating efficiency and selectivity in tumor cell killing.

### EXAMPLE 1

In Vivo Injection of Phage Display Library and Recovery of Organs and Phages:

Phage libraries are commercially available. Two of them sold by New England Biolabs were used. PhD-12 contains phages with random 12 amino acid sequences displayed by the pIII protein. PhD-12 stock titer is 1.3×10¹² pfu in 100 μl. Its complexity is 2.7×10⁹. PhD-C7C library displays random 7 mer amino acid sequences flanked by two cysteines displayed by the pIII protein. The PhD-C7C stock titer is 1.5×10¹² pfu in 100 μl with a complexity of 3.7×10⁹. Thus, injection of 5 μl of both libraries should contain at least 20 copies of each phage.

Before being injected into mice, 5 μl of PhD-12 phages were diluted in 200 μl of DMEM medium (Gibco BRL) (12-D) or in 200 μl of PBS (Dulbecco) (12-P). In parallel, 5 μl of PhD-C7C phages were diluted in 200 ul DMEM (7-D) or in 200 ul PBS (Dulbecco) (7-P).

Mice were anesthesized and the phage dilutions (12-D, 12-P, 7-D, 7-P) were injected through the tail vein (t=o min). Then, mice were perfused through heart with DMEM or PBS (t=2 min). Right after perfusion, and while in deep anesthesia mice were “snap-freezed” in liquid nitrogen.

Analysis is made on organ samples obtained from the injected animals that have been thawed partly at room temperature. Liver, heart, lung, spleen, kidney, and leg muscle were retrieved and placed into 1 ml of ice cold DMEM+PI or PBS+PI (PI is a protease inhibitor cocktail provided by Boehringer ref 1697498). Hearts and livers were lightly grounded with Polytron in an ice/water bath. The tissues were washed 3 to 5 times with 5-10 ml of ice cold DMEM+PI, 1% BSA, 0.1% Tween-20, or PBS+PI, 1% BSA, 0.1% Tween-20. Selected phages were eluted by competition with bacteria. For this purpose, recovered tissues were incubated with 1 ml of early log-phase E. coli ER2537 (New England Biolabs, ref 8110), 20 min at room temperature, with slow shaking. 10 ml of LB medium were added and the whole volume was incubated 20 min at room temperature, with shaking. An aliquote (10 μl) was used for phage titration (Maniatis, Laboratory Manual (1989), Cold Spring Harbor, Laboratory Press) whereas the rest was added to 10 ml of LB medium in an 250 flask. After addition of 150 μl of an overnight culture of ER2537, the culture was incubated 4.5 h with vigorous shaking at 37° C. The culture was centrifuged 10 min at 10 krpm (SS34) at 4° C. two times. 80% of the supernatant was collected and added to ⅙ vol (2.66 ml) of 20% (w/v) PEG-8000, 2.5 M NaCl. Phages were precipitated overnight at 4° C. in order to recover a concentrated stock of the selected phages that was subsequently titered according to the precited technique.

This selection protocol was done three times, before single plaques were picked for DNA isolation and sequencing.

From each round of selection described in example 1, the total number of recovered phages was calculated per 150 mg tissue from each of the organs. The recovered phages were titered before the amplification steps. When phages are recovered from a specific organ in a selection round, it is expected that the recovery from the same organ will increase in the next selection round, but the recovery should not increase from the other organs. FIG. 1 shows the results obtained from an in vivo selection targeting heart in Balb/c mice. Generally, an increase of the phage recovery from the target organ is observed for each selection round. After the three rounds of selection, fifty random phages were picked for sequencing. Table 2 represents a selection of peptides and their frequency of recovery within a selected phage pool. The number indicates the number of times the sequence was found/the number of sequences done in total in the particular experiment.

TABLE 2

| | |
|---|---|
| Peptide sequence | Frequency |

| | | |
|---|---|---|
| Heart Selection: | | |
| THPRFAR | (SEQ ID NO: 1) | 10/50 |
| HWAPSMYDYVSW | (SEQ ID NO: 78) | 12/50 |
| QTSSPTPLSHTQ | (SEQ ID NO: 4) | 5/50 |
| HLPTSSLFDTTH | (SEQ ID NO: 5) | 4/50 |
| YPSAPPQWLTNT | (SEQ ID NO: 10) | 4/50 |
| HVNKLHG | (SEQ ID NO: 16) | 3/50 |
| SGRIPYL | (SEQ ID NO: 19) | 3/50 |
| LSPQRASQRLYS | (SEQ ID NO: 21) | 3/50 |
| WKSELPVQRARF | (SEQ ID NO: 29) | 3/50 |
| HFTFPQQQPPRP | (SEQ ID NO: 36) | 3/50 |

| | |
|---|---|
| Peptide Selection | Frequency |

| | | |
|---|---|---|
| Tumor Selection: | | |
| TQSPLNYRPALL | (SEQ ID NO: 43) | 6/50 |
| THRPSLPDSSRA | (SEQ ID NO: 50) | 5/50 |
| SFPTHIDHHVSP | (SEQ ID NO: 55) | 4/50 |
| DAQQLYLSNWRS | (SEQ ID NO: 59) | 3/50 |
| P815: | | |
| MHNVSDSNDSAI | (SEQ ID NO: 64) | 4/50 |
| Liver Selection: | | |
| GHLIPLRQPSHQ | (SEQ ID NO: 79) | 6/50 |

### EXAMPLE 2

Analysis of Specificity of Candidate Phages in the Heart:

Stocks were made of these candidate phages and specificity tested in vivo by IV injection, recovery of target and control organs and calculation of total candidate phages recovered per gram tissue. Alternatively, candidate phages were injected with a negative control phage which yields white plaques instead of blue plaques. The ratio candidate/control recovery is then compared between target and control organs. In the literature, ratios of 2 to 35 have been described (Rajotte et al., J. Clin. Invest. 102 (1998) 430). FIG. 2 shows an example of in vivo testing of specificity of candidate phages with a co-injected negative control. The sequence of the displayed peptide is shown in the figure. In both cases, a higher recovery is found in the target organ (heart) than in the control organ.

### EXAMPLE 3

Incubation of Fixed Organs with Subtracted Phage Display Library and Recovery of Phages:

Subtraction: Phages were preincubated on non target cells, such as Hela (ATCC CCL-2) or 293 (ATCC CRL-1573). For this purpose, cells were grown to confluency in a flask (≧6.3×10⁶ cells) before being fixed in PBS, 0.05% glutaraldehyde for 10 min. The fixed cells were washed 5 times with PBS, 1% BSA to remove glutaraldehyde. 5 μl of the phage display library were diluted in PBS, 1% BSA (2.4 ml, or in smallest volume that covers the plate), added to the fixed cells and incubated 1 h at room temperature with slow rotation. The supernatant containing the subtracted phage suspension was collected by centrifugation 3 min at 1.5 krpm.

Preparation of subtractor cells in suspension: The cells were washed in PBS and detatched with 2 mM EDTA in PBS. After centrifugaion, the cells were resuspended in PBS, 0.05% glutaraldehyde, 1 mM MgCl₂ for 10 min. the cells were washed 5 times with PBS, 1% BSA, 1 mM MgCl₂, and stored in PBS, 1% BSA (6.3×10⁶ cells/ml or higher).

After anesthesia of a Balb/c mouse, organs were mildy fixed by total body perfusion with PBS, 0.05% glutaraldehyde for 10 min. Liver, heart, lung, spleen, kidney, and leg muscle were retieved. Liver and heart were minced with scissors and the fragments were kept at 4° C. in 1 ml PBS, 1% BSA in a polystyrene tube. The other organs were frozen at −80° C. The phage dilution mixed to 6.3×10⁶ subtractor cells was added and incubated overnight at 4° C. with slow rotation.

The supernatant was discarded and the organ fragments were washed with PBS, 1% BSA, 0.05% Tween-20 (5 times). The fragments were kept in 300 μl of wash buffer in a 15 ml tube and the selected phages were eluted at low pH by adding 450 μl of 50 mM Na-citrate, 140 mM NaCl, pH 2.0 for 5 min. Neutralization was made by adding 57 μl of 2 M Tris pH 8.7.

Titration was made on an aliquote (1-10 μl), and the rest of the supernatant was added to 20 ml of LB medium and 200 μl of an overnight culture of ER2537 before being incubated 4.5 h with vigorous shaking at 37° C. After two centrifugations 10 min at 10 krpm (SS-34) at 4° C., 80% of the supernatant was harvested to which ⅙ vol (2.66 ml) of 20% (w/v) PEG-800, 2.5 M NaCl was added. The mixture was precipitated overnight at 4° C. and the concentrated stock of the selected phages was recovered and titered.

This selection protocol is done three times, before single plaques are picked for isolation of DNA and sequencing.

FIG. 3 shows results obtained from an in vivo selection targeting liver and heart in Balb/c mice. Generally, an increase of the phage recovery from the target organ is observed for each selection round.

### EXAMPLE 4

Incubation of Target Cells with Subtracted Phage Display Library and Recovery of Phages:

Subtraction was done with cells that do not express the target molecules (e.g., MUC-1 polypeptide) as described above. However, the total unsubtracted phage display library may also be used.

The phage suspension was added to the target cells (non-fixed or fixed) and incubated (shortly or overnight) at 4° C. (or other temperature) with slow rotation. The supernatant was discarded and the cells were washed 5 times with PBS, 1% BSA, 0.05% Tween-20. The bound phages were eluted at low pH by adding 450 μl of 50 mM Na-citrate, 140 mM NaCl, pH 2.0 for 5 min. The 57 μl of 2 M Tris pH 8.7 was added to neutralize the phage solution.

Titration was made on an aliquote (1-10 μl), and the rest of the supernatant was added to 20 ml of LB medium and 200 μl of an overnight culture of ER2537. The mixture was incubated 4.5 h with vigorous shaking at 37° C. After two centrifugations 10 min at 10 krpm (SS-34) at 4° C., 80% of the supernatant was harvested, added to ⅙ vol (2.66 ml) of 20% (w/v) PEG-8000, 2.5 M NaCl, and precipitated overnight at 4° C. The selected phages were recovered as a concentrated stock, and titered.

This selection protocol was done three times, before single plaques were picked for isolation of DNA and sequencing.

4.1 Isolation of Peptides Exhibiting Specific Binding to MUC-1 Expressing P815 Tumor Cells:

P815 tumor cell binding phages were isolated by first performing three substractions on P815pAG60 (P815 cells transfected with a Neomycin expression cassette), and subsequently three selection-amplification cycles on P815MUC1 cells (P815 cells (ATCC TIB-64) transfected with MUC1 and Neomycin expression cassettes). P815 are mouse mastocytoma cells available at the ATCC collection (ATCC TIB-64). P815pAG60 cells were grown in DMEM supplemented with 10% fetal calf serum (FCS), 2 mM glutamine, 1 mM sodium pyruvate, 40 μg/ml gentamycin and non-essential amino acids. P815MUC1 cells were grown in the same medium with 1 mg/ml G418 to maintain the expression of the MUC1 gene.

For the three subtraction steps, 1×10⁷ P815pAG60 cells were incubated with 1.5×10¹¹ phages from the NEB phage libraries PhD-12 or PhD-C7C (catalog no. 8010 and 8020; NEB, Beverly, USA) for 1 hour at room temperature with slight agitation, in 1 ml of PBS-1% BSA. Cells were then collected by centrifugation at 2500 rpm for 3 minutes. An aliquot of the supernatant was kept for titration, and the rest was incubated again with 1×10⁷ P815pAG60 cells a second and a third time without amplification of the phage pools.

For the three selection cycles, the phage pool from the three successive subtractions were incubated with 5×10⁶ P815MUC1 cells for 4 hours at 4° C. with slight agitation, in 1 ml of PBS-1% BSA. The cells were then washed 5 times with 1 ml of PBS-1% BSA-0.1% Tween 20, and transferred to a new tube during the first wash and before elution. Phages bound to P815MUC1 cells were then eluted with 100 μl of 0.1M glycine-HCl pH2.2 for 10 minutes on ice, cells were pelleted by centrifugation, and the supernatant containing the eluted phage was neutralized with 10 μl of 2M Tris-HCl pH8.

Eluted phages were amplified in 20 ml of LB with 200 μl of an overnight culture of ER2537 bacteria (NEB) for 4.5 h at 37° C. under vigorous shaking. Then bacteria were removed by 2 centrifugation steps at 10000 rpm for 10 minutes, and to 16 ml of supernatant 2.33 ml of 20% PEG 8000, 2.5M NaCl was added for overnight precipitation of the phages at 4° C. The supplier's protocol was then followed to grow and titer a concentrated stock of phages. After the 3rd selection cycle on P815MUC1 cells, the ratio of recovered versus input phages increased by an enrichment factor of up to 500 for the selected pool.

32 single phages were isolated from the third selection pool, amplified and their genomes sequenced to deduce the amino acid sequence of their display peptide. The results are shown in Table 3. From the selection of the PhDC7C phages, two different peptide sequences were enriched and thus represented multiple times. In the selected PhD12 pool, five different phage sequences were identified.

TABLE 3

Sequence and frequencies of isolated
candidates:

| | |
|---|---|
| | Frequency of Recovery |
| Sequence | on P815MUC1 Cells |

| | | |
|---|---|---|
| CNDIGWVRC | (SEQ ID NO: 67) | 24/32 |
| CWPYPSHFC | (SEQ ID NO: 68) | 7/32 |
| MPLPQPSHLPLL | (SEQ ID NO: 69) | 11/32 |
| LPQRAFWVPPIV | (SEQ ID NO: 70) | 7/32 |
| WPVRPWMPGPVV | (SEQ ID NO: 71) | 5/32 |
| WPTSPWLEREPA | (SEQ ID NO: 72) | 2/32 |
| WPTSPWSSRDWS | (SEQ ID NO: 73) | 1/32 |

Two other phages were isolated using the technique as described above with the exception that elution was performed with an anti-MUC-1 antibody (12C10 which is a subclone of H23 hybridoma described in Keydar et al., 1989, PNAS, 86, 1362-1366). The sequences of the selected phages are CWPMKSLFC (WPM1) and CWPMKSQFC (WPM2).

4.2 Specific Binding of Selected Phages to P815 Cells:

The specificity of the selected phage candidates was then tested by incubating individual phages with P815 cells. Binding of candidates was compared to two negative control phages: empty M13 and GHL, a non-selected phage from the PhD12 library. These studies were performed on P815pAG60, P815MUC1 and non-transformed P815 cells using the phage titration assay or immunostaining by FACS (fluorescence activated cell sorting). The results demonstrate that the described selection scheme allowed the isolation of phages which bind specifically and with high affinity to P815 cells when compared to the negative controls.

Titration Assay:

1×10⁷ cells were incubated with 1.5×10¹¹ infectious particles from a selected candidate or control phage. Cells were washed and bound phages were eluted and titered as described above.

The results presented in FIG. 4 demonstrate that all candidates bound with at least 100-fold higher affinity to P815MUC1 and P815pAG60 cells than the controls. The WPY peptide exhibited the best affinity for P815 cells, followed by the NDI phage, and then the 12 amino acid peptide phages. All candidates, except WPY, showed at least 3 times higher binding to P815MUC1 than to P815pAG60 cells.

In addition, the candidate phages were incubated under similar experimental conditions with six other murine and human tumor cell lines: the murine carcinoma cell line RENCA (Murphy et al., 1973, J. Natl. Cancer Inst. 50, 1013-1025), a murine melanoma cell line B16 (ATCC CRL-6322), a human cervix carcinoma cell line HeLa (ATCC CCL-2), a human colorectal cancer cell line WiDr (ATCC CRL-218), and two human breast cancer cell lines MDA-MB-435 (ATCC HTB-129) and MDA-MB-231 (ATCC HTB-26) and their binding analysed by titration studies or FACS assays. All of these cell lines were grown in DMEM supplemented with 10% FCS, 2 mM glutamin and 40 μg/ml gentamycin. Except for WPY which exhibited a specific binding to RENCA cells with up to 10000-fold higher affinity than an M13 control phage, all other candidate phages bound these cell lines with the same affinity as an M13 control phage, indicating that they exhibit high specificity for certain tumor cells types, in particular lymphatic tumors. On the contrary, the WPY phage exhibits a high specificity for at least the two tumoral cell lines RENCA and P815 indicating that it may bind to several different tumor cell types.

FACS Assay:

5×10⁵ cells per well were placed in a 96-well plate, 10¹¹ phages were added and incubated for 2 hours at 4° C. under shaking. Cells were washed 4 times with 150 μl of FACS buffer (PBS with 1% BSA, 0.1% human γ-globulin, 5 mM EDTA). 100 μl of an anti-fd bacteriophage antibody (Sigma, St Louis, USA; catalog no: B7786) at 1/500 were added to the wells and incubated for 45 minutes at 4° C. Cells were washed 4 times with FACS buffer and incubated for 45 minutes at 4° C. with a goat anti-rabbit IgG (H+L) antibody coupled to FITC (Biotechnology Associates, Birmingham, USA; catalog no: 4052-02) at 1/200. Cells were washed 4 times with FACS buffer and the fluorescence measured with a FACScan (Becton Dickinson, San Jose, USA). The results were analyzed with the Cellquest software.

The specificity of the above described candidates compared to empty M13 was confirmed by FACS analysis on P815 cells. The WPY, MPL and LPQ phages showed high specific binding to P815MUC1 cells as well as to the original non-transfected P815 cell line. All other clones exhibited binding to P815MUC1 cells, but differed in their binding to non-transfected P815 cells.

4.3. Specific Binding of the WPY and LPQ Synthetic Peptides to P815MUC1 Cells:

Synthetic peptides corresponding to the WPY and LPQ sequences of the previously selected phages (second and fourth sequences of Table 3) were synthetized (Neosystem, strasbourg, France). Increasing amounts of WPY peptide (0.1, 10 and 500 μM) and LPQ peptide (0.1, 10 and 1000 μM) or control peptides GHL and SGR (a non-selected phage from the PhD-C7C library) were diluted in a total volume of 1 ml of PBS-BSA1 % with 5.10⁶ P815MUC1 cells and incubated for 1 hour at 4° C. under slight agitation. 1.10¹⁰ WPY or LPQ phages in 200 μl of PBS-1% BSA were added and incubated with the cells and the peptide for two hours at 4° C. under slight agitation. Cells were then washed and bound phages eluted and titered following the same protocol as for the selections. The WPY and LPQ peptides were able to inhibit in a dose-dependant manner the binding of the corresponding phages, whereas the control peptides did not significatively inhibit WPY and LPQ phage binding, showing that the synthetic peptides efficiently compete for the binding of the phages displaying the same sequence. These resultes indicate that synthetic peptides representing the WPY and LPQ sequences also exhibited specific binding to P815 MUC-1 cells.

Interestingly, the WPY peptide (500 μM concentration) did not significatively inhibit the binding of the LPQ phage, indicating that these two peptides recognize different molecular targets.

### EXAMPLE 5

Isolation of Phages Exhibiting Specific Binding to WiDr (Human Colorectal Carcinoma Cells):

All cells originated from the ATCC collection and were maintained in DMEM, supplemented with 10% fetal calf serum (FCS), 2 mM glutamine, and 40 μg/ml gentamycin.

The supplied PhD-12 or PhD-C7C library was first preadsorbed on HeLa cells three times before the first selection on WiDr cells. The HeLa cells were brought in suspension by incubating in PBS, 10 mM EDTA. The cells were then washed twice by adding 10 ml PBS, and collected by centrifugation (2500 rpm for 3 min). The cells were counted and resuspended in 1 ml PBS, 1% BSA, per 10⁷ cells.

1.5×10¹¹ pfu from the phage library were added to 1 ml of cells, and the mix incubated for 1 h at room temperature, with slow shaking. After centrifugation at 2500 rpm for 3 min, the supernatant was incubated again with 10⁷ HeLa cells. This subtraction protocol was repeated 3 times. The final supernatant (the subtracted pool of phages) was then incubated with 5×10⁶ WiDr cells in suspension for 4 hours at 4° C., with slow shaking. The cells were washed five times in 1 ml cold PBS, 1% BSA, 0.1% Tween-20, and collected as above. The bound phages were eluted by adding 100 μl 0.1 M Glycine-HCl, pH 2.2, and incubating 10 min on ice. After centrifugation at 2500 rpm for 3 min, the supernatant was neutralized with 10 μl 2 M Tris-HCl, pH. An aliquot of 10 μl was titered and the rest was amplified by adding the eluted phages to 20 ml LB with 200 μl overnight E. coli ER2537 culture. The culture was incubated with strong agitation for 4 h and phage purification was performed according to the providers protocol (NEB).

The selection on WiDr cells was repeated 5 times in total, either with no subtraction before the 2^{nd} to 5^{th} selection, or with 3 subtractions on 293 cells before each selection. Twenty four single phages from the final selected pools were amplified and sequenced to identify the peptide sequence.

TABLE 4

| Subtraction/ | | | |
|---|---|---|---|
| Selection | Cells | Sequence | Frequency |
| 1^{ST} Subtraction | HeLa WiDr | HEWSYLAPYPWF | 13 of 24 |
| 1^{st} Selection | 293 WiDr | (SEQ ID NO: 74) | |
| 2^{nd}-5^{th} Subtraction | | | |
| 2^{nd}-5^{th} Selection | | | |
| 1^{st} Subtraction | HeLa WiDr | QIDRWFDAVQWL | 24 of 24 |
| 1^{st}-5th Selection | | (SEQ ID NO: 74) | |
| 1^{ST} Subtraction | HeLa WiDr | CLPSTRQTC (SEQ | 24 of 24 |
| 1^{st} Selection | 293 WiDr | ID NO: 74) | |
| 2^{nd}-5^{th} Subtraction | | | |
| 2^{nd}-5^{th} Selection | | | |

The specificity of the selected phages was tested by binding to WiDr cells in comparison to the binding of the M13 wild type phage, and in comparison to the binding to different tumor cells lines. The binding was done as described above for the selection.

FIG. 5 shows output/input ratios of the HEWSYLAPYPWF phage when binding was tested on different cells, compared to the M13 wild type phage. The HEW phage shows a 1900-fold higher affinity to WiDr cells than the M13 wild type phage, and a 270-fold higher affinity to MDA-MB-435 cells, while the affinity to 293, and HeLa cells is similar to the M13 wild type affinity.

In a parallel experiment, five selections were made on WiDr cells, but subtraction of the library was only done on HeLa cells before the first selection on the WiDr cells. Selected phages were collected after each of the five rounds of WiDr cell selection (pool 1 to pool 5). From the fifth pool, 24 single plaques were amplified and the insert, corresponding to the peptide, was sequenced. The QIDRWFDAVQWL sequence (SEQ ID NO: 75) was obtained from all phages. The purified [symbol] QID [symbol] -phage, and the fith pool, was found to have affinity to various tumor cell lines, in contrast, the unselected pool 1 did not show affinity to the tumor cell lines. These results demonstrate an affinity of the QID phage to several different tumor cell types.

The same protocol as for selection of the [symbol] HEW [symbol] -phage was repeated with the pHD-C7C library. The fifth pool from this selection contained phages displaying the sequence CLPSTRWTC (SEQ ID NO: 76) and showed specific binding to WiDr cells compared to the subtractor 293 cells.

### EXAMPLE 6

Construction of Adenoviral Vectors for Tuor-Cell-Specific Targeting:

Abstract:

One of the main limitations of adenoviral vectors for cancer gene therapy applications is their poor tropism for many tumor tissues. To overcome this problem, we introduced into the knob domain of the viral fiber capsid protein a tumor-targeting peptide, identified by phage display on whole cells. We show that this peptide specifically directs the tropism of recombinant adenoviral vectors to several colon and breast human cancer cell types, by providing a novel, peptide-mediated, entry pathway. Moreover, combined with the CAR-ablating S408E mutation of the fiber protein, high level of infection is maintained only in target tumor cells, showing that the HEW pathway is active in the context of a CAR-deficient pathway. In conclusion, adenoviral vectors carrying the HEW peptide could be useful for gene delivery into HEW-targeted tumor tissues that express low levels of Ad natural receptors.

Introduction:

Recombinant adenovirus (Ad) represent promising gene therapy vectors, owing to their high efficiency of infection on many dividing and quiescent cells. However, for cancer gene therapy, this broad tropism may be a disadvantage. Moreover, many tumor cells express low levels of CAR, the natural Ad receptor (Li et al., Cancer Res 59 (1999), 325-330; Hemmi et al., Hum Gene Ther 9 (1998), 2363-2373; Miller et al., Cancer Res 58 (1998), 5738-5748), and are hence poorly transduced by Ad vectors. Therefore, specific Ad vector targeting to tumor tissues would lead to a significantly improved efficacy, thereby increasing the therapeutic index.

Because of the ubiquitous expression of Ad receptors (Pimental et al., J Biol Chem 271 (1996), 28128-28137), abolition of the natural tropism is a first step necessary for the construction of a tumor-specific vector. The Ad fiber protein is involved in the primary binding of Ad to one of its cellular receptors, CAR (Bergelson et al., Science 275 (1997), 1320-1323). Therefore, point mutations were introduced into the fiber knob, that abolished the interaction with CAR without disturbing the structure of the fiber. Several CAR binding-ablated mutant vectors have been described (Bewley et al., Science 286 (1999), 1579-1583; Roelvink et al., Science 286 (1999), 1568-1571; Kirby et al., J Virol 73 (1999), 9508-9514; Kirby et al., J. Virol. 74 (2000), 2804-2813; Leissner et al., Gene Ther. 8 (2001), 49-57; Jakubczak et al., J. Virol. 75 (2001), 2972-2981), but the biodistribution of such CAR-deficient vectors was not modified after intravenous injection (Jakubczak et al., J. Virol. 75 (2001), 2972-2981). This observation suggests that other receptors might be involved in Ad tropism in vivo, such as Heparan sulfate Glycosaminoglycans (Dechecchi et al., J. Virol. 75 (2001), 8772-8780) or sialic acid (Arnberg et al., J. Virol. 74 (2000), 42-48).

Concerning the introduction of a new tropism, small peptide ligands were successfully introduced either at the carboxy-terminus of the fiber knob (Wickham et al., Nat Biotechnol. 14 (1996), 1570-1573; Yoshida et al., Hum Gene Ther. 9 (1998), 2503-2515; Bouri et al., Hum Gene Ther. 10 (1999), 1633-1640), or in the HI loop (Krasnykh et al., J. Virol. 72 (1998), 1844-1852; Dmitriev et al., J. Virol. 72 (1998), 9706-9713; Nicklin et al., Mol Ther. 4 (2001), 534-542) and shown to be accessible for binding to their receptor. However, very few studies showed specific targeting of Ad vectors to cancer cells (Turunen et al., Mol Ther. 6 (2002), 306-312).

We recently identified a tumor-binding peptide by phage display on whole cells (Rasmussen et al., Cancer Gene Ther. 9 (2002), 606-612). The HEWSYLAPYPWF-displaying phage was selected on human colorectal cancer cells, and showed more than 1000-fold higher binding efficiency for WiDr cells when compared to five other human cancer cell lines and to wild-type M13 phage. Specific binding to the MDA-MB435 breast cancer cell line was also observed. Moreover, the free peptide was able to specifically compete the binding of the corresponding phage, indicating that the specificity of the peptide is independent of the display by the phage pIII coat protein.

Here, we report the phenotype of Ad vectors with the HEW peptide genetically engineered into the HI loop, in the absence or presence of additional CAR-ablating mutation, leading to specific and efficient targeting to several tumor cell types.

Materials and Methods:

Cells:

The human embryonic kidney cell line 293 and the human cancer cell lines (WiDr, SW480, LOVO, Caco2: colon carcinoma; MDA-MB435, MCF-7, MDA-MB231, T47D: breast carcinoma; HeLa: cervix carcinoma; A549: lung carcinoma; HepG2: hepatocarcinoma) were obtained from the American Type Collection of Cells (ATCC, Rockville, Md., USA). They were grown at 37° C. in DMEM supplemented with 10% fetal calf serum and antibiotics. HUVEC (Human Umbilical Vein Endothelial Cells, ATCC) were grown in Endothelial Cell Growth Medium, containing 2% FCS, 0.1 ng/ml EGF, 1 ng/ml bFGF, 1 μg/ml hydrocortison and antibiotics.

293-Fiber cells (293-Fb), which constitutively express the adenovirus type 5 fiber protein, have been described previously (Legrand et al., J. Virol. 73 (1999), 907-919).

Construction of Fiber-Modified Viral Genomes:

All cloning steps were performed using standard molecular biology techniques (Sambruck et al., Molecular Cloning; A Laboratory Manual (2001), Cold Spring Harbor Laboratory Press, Cold Spring Harbor N.Y.) or according to the manufacturer's recommendations when a commercial kit is used. In order to introduce peptides in the Ad5 fiber knob, a NarI or a SpeI restriction site was first introduced with the Sculptor in vitro mutagenesis system (Amersham, Les Ullis, France) in fiber gene-containing M13 templates (M13F5knob and M13F5-S408E-knob (Leissner et al., Gene Ther. 8 (2001), 49-57), at the 3′ extremity or in the HI loop, respectively, using the following antisense oligonucleotides: OTG14499: 5′-cgattctttagctgccgggcgccgaggcggaggcggaggcg-3′ (SEQ ID NO: 80) and OTG14509: 5′-catagagtatgcacttggact agtgtctcctgtttcctgtg-3′ (SEQ ID NO: 81). The 1.1 Kb EcoRI-BamHI fiber gene fragment containing the NarI site was subcloned into pBSK, because a NarI site already existed in the M13 sequence. Complementary oligonucleotides coding for the HEW peptide flanked by NarI extremities (5′-cgcccggcagccacgagtggagctacctggccccctacccatggttctaagg-3′ (SEQ ID NO: 82) and 5′-cgccttagaaccatgggtagggggccaggtagctccactcgtggctgccggg-3′ (SEQ ID NO: 83)) or by SpeI extremities (5′-ctagtcacgagtggagctacctggccccctacccatggttca-3′ (SEQ ID NO: 84) and 5′-ctagtgaaccatgggtagggggccaggtag ctccactcgtga-3′ (SEQ ID NO: 85)) were then annealed and ligated into NarI-linearized pBSK-fb and pBSK-S408E-fb, or into SpeI-linearized m13F5knob and m13F5-S408E-knob plasmids, respectively. The 1.1 Kb SmaI fragments containing the modified fibers were then directly introduced by homologous recombination into the BstBI-restricted AdE1° [CMVLacZ]E3° (Fong et al., Drug Dev. Res. 33 (1994), 64-70). Virus production and titration were performed as described in Leissner et al. (Gene Ther. 8 (2001), 49-57).

Western Blot Analysis:

Viral particles (10¹⁰) were diluted in 2× Laemmli buffer, incubated for 5 min at 95° C., loaded onto a NuPAGE 4-12% Bis-Tris gel (Invitrogen), and then transferred to nitrocellulose. Filters were hybridized with either anti-knob or anti-penton base rabbit polyclonal antibodies (Legrand et al., J. Virol. 73 (1999), 907-919). Bound antibodies were detected by using a horseradish peroxidase-conjugated donkey anti-rabbit antibody according to the instructions of the manufacturer (ECL detection kit; Amersham, Les Ullis, France).

Infection Experiments:

Cell culture monolayers were incubated in 100 μl of DMEM-2% FCS with control (Ad-fbwt or AdS408E) or HEW-displaying (Ad-HEW and AdS408E-HEW) viruses at increasing particle/cell ratios for 30 minutes at 37° C., followed by addition of 1 ml of DMEM-2% FCS medium. After a 24 hour-incubation, cells were either fixed and stained with X-gal for detection of LacZ expression, or lysed in RLB buffer (Promega, Lyon, France) for monitoring of β-galactosidase activity, using a chemiluminescent substrate (luminescent β-galactosidase detection kit, Clontech, Palo Alto, Calif., USA).

Competition Experiments:

Cell culture monolayers were incubated for 30 minutes at 37° C. with either medium or competitor molecules. Control and targeted viruses were then added and the infection efficiency was determined by total β-galactosidase activity as described above. As competitor for the normal adenoviral entry process, purified Ad5 knob (10 μg/ml) was used. The potential entry of targeted viruses through an HEW-specific pathway was assessed by using free HEW peptide, fused to polylysin (Rasmussen et al., Cancer Gene Ther. 9 (2002), 606-612), as competitor (0.3 or 3 μg/ml).

Results:

Construction of HEW-Displaying Viruses:

The HEW peptide was previously shown to bind specifically to WiDr cells on the phage surface and as soluble peptide fused to polylysin (Rasmussen et al., Cancer Gene Ther. 9 (2002), 606-612). In both cases, its amino-terminal extremity was free. Two positions can be used for insertion of this ligand in the Ad fiber knob, the carboxy-terminal extremity or the HI loop respectively. For the purpose of the invention, the HEW peptide was inserted in the HI loop of the Ad fiber knob and its targeting capacity evaluated in different cell models. Oligonucleotides encoding the peptide sequence were inserted into the HI loop between amino acids 545 and 546, of wild-type or S408E (Leissner et al., Gene Ther. 8 (2001), 49-57) mutant fibers (FIG. 6). Virus particles were produced on human 293 cells (2×10⁸) infected with the different viruses at 1 IU (infectious unit)/cell. At 40 to 72 hours after infection, cells were recovered and viruses were extracted and purified. Particles and IU titers were determined as described previously (see for exemple Leissner et al., 2001, Gene Ther. 8, 49-57). Viruses containing the peptide in the HI loop were readily produced and could be amplified to high titers. Indeed, as shown in Table 5, large-scale production of HI loop-displayed HEW viruses (Ad-HEW and AdS408E-HEW) yielded particles and infectious units (IU) titers equivalent to control Ad (Ad fbwt and AdS408E vectors respectively). These results suggested that the insertion of HEW in the HI loop did not perturb the Ad fiber structure, resulting in efficient virus assembly and production.

TABLE 5

| Virus | Particles/ml | IU/ml | IU/particles |
|---|---|---|---|
| Ad-wt | 4.21 × 10¹² | 2.83 × 10¹² | 1/149 |
| Ad-HEW | 2.65 × 10¹² | 1.66 × 10¹² | 1/160 |
| AdS408E | 8.61 × 10¹² | 8.21 × 10⁸ | 1/10487 |
| AdS408E-HEW | 2.19 × 10¹² | 6.45 × 10⁷ | 1/33876 |

Furthermore, the amounts of modified fibers present into purified virions were equivalent to non-modified fibers, indicating that HEW-containing viruses incorporated stoichiometric amounts of fiber. Thus, the introduction of the HEW peptide into the HI loop did not perturb virus growth, nor correct incorporation of the modified fibers into the capsid.

Infection of Non-Target Cells by HEW Viruses:

The infectivity of the modified viruses was determined on non-target 293 cells. For this purpose, 2×10⁵ 293 cells in monolayers were infected with control and HEW-containing viruses at the same particles/cell ratios (varying from 1 to 10⁴). At 20 hours post-infection, cells were fxed and stained for β-galactosidase expression (FIG. 7A). The number of infected cells was determined by counting blue (β-galactosidase-positive) cells, as an evaluation of the transduction efficiency (FIG. 7B). Alternatively, cells infected with high viral titers were lysed and β-galactosidase activity of the supernatant was monitored using a chemiluminescent detection kit (FIG. 7C).

As shown in FIG. 7, the transduction efficiency and the transgene expression level provided by the HEW-containing and control vectors were comparable, both in the context of wild-type and CAR deficient fiber. Therefore, these results confirm that the HEW peptide does not have any positive or negative influence on the infectivity for non target cells such as 293 cells. Similar results were obtained with HeLa cells, to which the HEW phage did not show any specific binding.

Infection of Target Cells by HEW Viruses:

The HEW phage was previously shown to specifically bind to the WiDr colorectal cancer cells used for the selections, as well as to a breast cancer cell line (MDA-MB435) (Rasmussen et al., Cancer Gene Ther. 9 (2002), 606612). In order to determine if this specificity was conserved in the modified Ad vectors, and if it could lead to improved infection efficiency, target cells were infected with the different viruses, and transduction efficiency and transgene expression levels were determined.

WiDr Cells:

2×10⁵ WiDr cells were infected with control (Ad-fbwt and Ad-S408E) and HEW-containing viruses (Ad-HEW and AdS408E-HEW) at the same particles/cell ratios (varying from 1 to 10⁴). At 20 hours post-infection, cells were fixed and stained for β-galactosidase expression (FIG. 8A). The number of infected cells was determined by counting blue cells (FIG. 8B). Alternatively, cells infected with high viral titers were lysed and β-galactosidase activity of the supernatant was monitored using a chemiluminescent detection kit (FIG. 8C). At 100 P/cell, the numbers of blue-stained cells showed a 2.5-fold higher infectivity for Ad-HEW compared to Ad-fbwt (FIG. 8B). Most interestingly, AdS408E-HEW was 30-fold more infectious than the non-targeted Ad-S408E vector, and showed the same transduction efficiency as Ad-fbwt. Thus, the decrease in infectivity caused by the ablation of CAR-mediated entry pathway is completely compensated for by the presence of HEW peptide in the fiber. The differences in transgene expression levels between control and HEW-containing viruses (FIG. 8C) confirmed that, for WiDr cells, HEW-displaying viruses are more potent gene transfer vectors than non targeted control vectors.

MDA-MB435 Cells:

As specific binding of the HEW peptide to the MDA-MB435 cell line was also observed, infection capability of control and HEW-containing viruses was determined with respect to this cell line. For this purpose, 2×10⁵ MDA-MB435 cells were infected with control (Ad-fbwt and Ad-S408E) and HEW-containing viruses (Ad-HEW and AdS408E-HEW) at the same particles/cell ratios (varying from 1 to 104). At 20 hours post-infection, cells were fixed and stained for β-galactosidase expression (FIG. 9A). The number of infected cells was determined by counting blue cells (FIG. 9B). Alternatively, cells infected with high viral titers were lysed and β-galactosidase activity of the supernatant was monitored using a chemiluminescent detection kit (FIG. 9C). It has to be noted that the MDA-MB435 cell line is hardly infected by Ad. This may be explained by the absence of CAR expression on the surface of this cell line, as shown by FACS analysis. In agreement with this observation, no blue staining was visible in wells infected with either Ad-fbwt or Ad-S408E viruses, even at 10⁴ P/cell, whereas HEW viruses yielded blue cells (FIG. 9A). To quantify the efficiency of infection, the target cells were then infected at P/cell ratios that yielded about the same number of blue cells (200 P/cell for targeted viruses, and 5000 P/cell for control viruses, FIG. 9B), revealing that both HEW-displaying viruses were 25-fold more infectious than control viruses on these target cells. In parallel, the level of gene expression increased by 15-fold for AdS408E-HEW infected cells compared to AdS408E infected cells, and by 30 to 40-fold for Ad-HEW infected cells compared to Ad-fbwt infected cells (FIG. 9C). Therefore, the infection level of this tumor cell line by Ad vectors is significantly improved in presence of the HEW peptide on the surface of viral particles.

Entry Pathway of HEW-Displaying Viruses:

To estimate the contribution of CAR-mediated infection for both control and targeted HEW-containing viruses, soluble knob was used in competition experiments with Ad-fbwt and Ad-HEW vectors, in control and target cells. For the study of target peptide-mediated entry, soluble HEW-K16 peptide was used as competitor.

In the non-target 293 and HeLa cell lines, both Ad-fbwt and Ad-HEW viruses were more than 90% inhibited by soluble knob, indicating that the CAR pathway represents the major entry pathway for these cells (FIG. 10A). This result also confirms that the presence of the HEW peptide in the HI loop does not prevent fiber interaction with its natural receptor in CAR-positive cells. On the other hand, consistent with the fact that these cells are not target cells of the peptide, the HEW-K16 free peptide had no effect on the transduction efficiency of Ad-fbwt and Ad-HEW (FIG. 10A), nor of Ad-S408E and Ad-S408E-HEW viruses (FIG. 10B), in these cell lines.

On WiDr cells, Ad-fbwt infection was efficiently competed by soluble knob, showing that the CAR pathway is important in this cell type. However, the infectivity of Ad-HEW was only marginally inhibited by soluble knob, suggesting the existence of an alternative entry pathway for this virus (FIG. 10C). In the presence of free HEW-K16 peptide, the transduction efficiency of Ad-HEW, but not of Ad-fbwt, decreased by 30%. Finally, the infection of WiDr cells by Ad-HEW could only be efficiently blocked by the presence of both knob and HEW-K16 peptide competitors (FIG. 10C). Thus, the Ad-HEW vector is able to enter WiDr cells either via interaction with CAR, or via interaction involving a receptor for the HEW peptide.

In the context of the S408E fiber mutation, the CAR pathway is abolished, and the soluble HEW-K16 peptide alone is sufficient to compete the infectivity of AdS408E-HEW virus (FIG. 10D). Hence, the entry of this virus in WiDr cells seems to be mediated mainly by interactions of the knob-displayed HEW peptide with a cell surface-expressed receptor of this peptide, thus explaining its better infectivity compared to the non targeted AdS408E virus.

Similar competition experiments were performed with MDA-MB435 cells. The soluble knob protein was not able to compete the infection of Ad-fbwt, thereby confirming the absence of CAR-mediated infection in this cell type (FIG. 10C). In the presence of soluble HEW-K16, Ad-HEW and AdS408E-HEW infection levels were 50% inhibited (FIGS. 10C and 10D). These results suggest that the HEW peptide, when inserted in the fiber knob, provides an efficient entry pathway to Ad vectors in CAR-negative MDA-MB435 cells, allowing to significantly improve the gene transfer efficiency.

Taken together, these results demonstrate that a new, specific entry pathway is provided by the interaction between the HEW peptide and its receptor on the surface of target WiDr and MDA-MB435 cells.

Infection of Other Tumor and Normal Cell Types by HEW Viruses:

In order to evaluate the specificity of the HEW peptide, the infection efficiency and entry pathway of Ad-HEW and AdS408E-HEW were determined on human cells originating from different tumors. A total of 12 cell lines were assayed, and the results show that in addition to WiDr and MDA-MB435 cells, two other cell types, colon carcinoma HT-29 cells and breast carcinoma MCF-7 cells respectively, were specifically targeted by the HEW-containing vectors, resulting in a 25 to 50 fold higher infectivity than control vectors (AdS408E being the control for AdS408E-HEW and Ad-fbwt being the control for Ad-HEW).

In these two cell types, AdS408E were significantly less infectious than Ad-fbwt, and Ad-fbwt was efficiently competed by soluble knob. These observations suggest that infection of these cells is CAR-dependent. The incorporation of the HEW peptide in the S408E fiber mutant vector induced a significant increase of infectivity, restoring a level of infection comparable to wild-type fiber vector. Moreover, competition assays with soluble HEW-K16 showed the existence of an HEW receptor-mediated entry pathway in HT-29 and MCF-7 cells. Moreover, this pathway could efficiently replace the CAR pathway, as indicated by the absence of knob competition of Ad-HEW.

Infection of A549 lung carcinoma cells yielded intermediate results: an increase of infectivity by a factor of 5 to 10 could be observed in the presence of HEW peptide in the fiber knob. However, the competition assays suggested that the CAR pathway is the dominant entry pathway for Ad-HEW.

The other studied cell lines represented two breast cancer cell line (MDA-MB231 and T47D), three colon carcinoma cell lines (LoVo, SW480 and Caco2), and one hepatoma cell line (HepG2). All of them showed infection and competition patterns similar to those observed with 293 and HeLa cells. These results show that the presence of HEW peptide on the virus surface does not have an influence on virus transduction, and suggested the absence of an HEW receptor-mediated entry in these cell lines.

We were also interested in determining the effect of HEW peptide on normal cells. Human primary endothelial cells (HUVEC) were chosen, as they would be in primary contact with the virus after intravenous injection. No increase of infectivity and no competition with HEW-K16 soluble peptide could be observed. These results suggest the absence of a specific tropism of HEW-containing viruses for this cell type.

Discussion:

In this study, a phage display-derived peptide, HEW, was incorporated into the Ad fiber protein in order to confer a new, tumor-specific tropism to adenoviral gene transfer vectors. We demonstrate that the 12 amino acids linear peptide could be introduced in the HI loop of the fiber knob without perturbing the incorporation of the protein in the capsid of purified virions, and with normal virus production yields. The infectivity of these HEW-displaying viruses on the producer cells 293 was identical to that of control viruses.

The HEW peptide was identified by phage display on WiDr colon cancer cells, and subsequent binding experiments on different cell types revealed a specific binding to WiDr as well as to MDA-MB435 breast cancer cells (Rasmussen et al., Cancer Gene Ther. 9 (2002), 606-612). Therefore, the transduction efficiency of HEW-displaying Ad was first studied with these two cell lines. The results clearly showed a specific increase of infectivity of HEW-containing viruses on target cells, which could be specifically inhibited by soluble HEW-K16 competitor peptide, thus suggesting the existence of an HEW-mediated entry pathway.

In order to further analyze the specificity of HEW-containing viruses, infection of several other human tumor cell lines was studied. No targeting of cervix carcinoma HeLa cells, hepatocarcinoma HepG2 cells, colon carcinoma SW480, LoVo and Caco2 cells, and of breast carcinoma MDA-MB231 and T47D cells could be observed, demonstrating that the expression pattern of the molecular target of the HEW peptide is very specific to certain cell types. Interestingly, several of these cell types were previously tested for HEW phage specificity study (293, HeLa, SW480, LoVo, MDA-MB231), and also showed an absence of specific binding to this phage (Rasmussen et al., Cancer Gene Ther. 9 (2002), 606-612). Therefore, these results also demonstrate that the cell specificity of a peptide can be conserved from phage display to Ad fiber display.

Two other colon and breast tumor cell lines, HT-29 and MCF-7, were identified as specific targets for the HEW peptide-displaying Ad vectors. Therefore, these results indicate that the molecular target of the HEW peptide is expressed on several, although not all, types of human colon and breast cancer cells. In addition, normal HUVEC primary endothelial cells did not show increased infection by HEW-containing vectors compared to control vectors.

Taken together, our results demonstrate that the introduction of a linear peptide in the HI loop of Ad fiber knob can specifically and efficiently redirect the tropism of Ad vectors towards particular tumor cell lines, thereby generating a new type of adenoviral vectors, that show a tumor cell-specific infectivity. Particularly, the AdS408E-HEW vector is 100-fold less infectious than wild-type fiber-displaying virus on CAR-positive non target cells, whereas its infection efficiency is equivalent to Ad-fbwt on WiDr, HT-29 and MCF-7 cells, and 30-fold higher on MDA-MB45 cells. Therefore, in a CAR-negative cellular context, a situation often encountered in primary tumor tissues, the HEW peptide might be useful to improve the delivery of therapeutic genes to tumor cells that express the molecular target of the HEW peptide. The subsequent introduction of targeting peptides, like the HEW peptide, in such detargeted vectors would then provide tumor cell-restricted vectors.

Each patent, patent application and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A peptide selected from the group consisting of:

| | |
|---|---|
| X₁LSPQRASQRLYSX₂ | (SEQ ID NO: 21) |
| X₁WKSELPVQRARFX₂ | (SEQ ID NO: 29) |
| X₁CNDIGWVRCX₂ | (SEQ ID NO: 67) |
| X₁CWPYPSHFCX₂ | (SEQ ID NO: 68) |
| X₁MPLPQPSHLPLLX₂ | (SEQ ID NO: 69) |
| X₁LPQRAFWVPPIVX₂ | (SEQ ID NO: 70) |
| X₁WPVRPWMPGPVVX₂ | (SEQ ID NO: 71) |
| X₁WPTSPWLEREPAX₂ | (SEQ ID NO: 72) |
| X₁WPTSPWSSRDWSX₂ | (SEQ ID NO: 73) |
| X₁HEWSYLAPYPWFX₂ | (SEQ ID NO: 74) |
| X₁QIDRWFDAVQWLX₂ | (SEQ ID NO: 75) |
| X₁CLPSTRWTCX₂ | (SEQ ID NO: 76) |
| X₁CWPMKSX₅FCX₂ | (SEQ ID NO: 77) |

wherein each X₁ and X₂ independently of one another represents any amino acid sequence of n amino acids, n varying from 0 to 50 and n being identical or different in X₁ and X₂, and wherein X₅ represents any amino acid.

2. A method for targeting a target cell, comprising targeting such cell with a peptide as defined by claim 1.

3. A heart targeting peptide comprising at least a three amino acid motif selected from the group consisting of:
SPQ, QRA, QRL or PQR, or any combination thereof and
SEL or PVQ or SEL and PVQ.

4. A heart targeting peptide according to claim 3, having the following sequence X₁LSPQRASQRLYSX₂ (SEQ ID NO: 21) or X₁WKSELPVQRARFX₂ (SEQ ID NO: 29), wherein each X₁ and X₂ independently of one another represents any amino acid sequence of n amino acids, n varying from 0 to 50 and n being identical or different in X₁ and X₂.

5. A tumor targeting peptide comprising at least a three amino acid motif selected from the group consisting of:
NDI, WPY, MPL, PSH, LPQ, WPV or WPT or any combination thereof, and
HEW, QID, WPM or CLP or any combination thereof.

6. A peptide according to claim 5, having the following sequence

| | |
|---|---|
| X₁CNDIGWVRCX₂, | (SEQ ID NO: 67) |
| X₁CWPYPSHFCX₂, | (SEQ ID NO: 68) |
| X₁MPLPQPSHLPLLX₂, | (SEQ ID NO: 69) |
| X₁LPQRAFWVPPIVX₂, | (SEQ ID NO: 70) |
| X₁WPVRPWMPGPVVX₂, | (SEQ ID NO: 71) |
| X₁WPTSPWLEREPAX₂, | (SEQ ID NO: 72) |
| X₁WPTSPWSSRDWSX₂, | (SEQ ID NO: 73) |
| or | |
| X₁HEWSYLAPYPWFX₂ | (SEQ ID NO: 74) |
| X₁QIDRWFDAVQWLX₂ | (SEQ ID NO: 75) |
| X₁CLPSTRWTCX₂ | (SEQ ID NO: 76) |
| X₁CWPMKSX₅FCX₂ | (SEQ ID NO: 77) |

wherein each X₁ and X₂ independently of one another represents any amino acid sequence of n amino acids, n varying from 0 to 50 and n being identical or different in X₁ and X₂ and wherein X₅ represents any amino acid.

7. A method for targeting to a heart cell, comprising targeting such cell with a peptide as defined by claim 3 or 4.

8. A method for targeting to a tumor cell, a metastasis or a tumor vasculature, comprising targeting such cell, metastasis or vasculature with a peptide as defined by claim 5 or 6.

9. A method for targeting to a colorectal tumor cell, comprising targeting such cell with a peptide as defined by claim 6 selected from the group consisting of X₁HEWSYLAPYPWFX₂ (SEQ ID NO: 74), X₁QIDRWFDAVQWLX₂ (SEQ ID NO: 75) and X₁CLPSTRWTCX₂ (SEQ ID NO: 76).

10. A method for targeting to a carcinoma tumor cell, comprising targeting such cell with a peptide X₁CWPYPSHFCX₂ (SEQ ID NO: 68) as defined by claim 6.

11. A composition comprising at least one peptide according to claim 1 and at least one therapeutic agent or alternatively at least one nucleic acid molecule encoding a peptide according to claim 1 and at least one therapeutic agent.

12. The composition according to claim 11, wherein said therapeutic agent is a vector for delivering at least one gene of interest to a target cell of a vertebrate.

13. The composition according to claim 12, wherein said vector is a plasmid, a synthetic or a viral vector.

14. The composition according to claim 13, wherein said viral vector is an adenoviral vector.

15. The composition accoding to claim 14, wherein said adenoviral vector is replication-defective.

16. The composition according to claim 11, wherein said peptide is operably coupled to said therapeutic agent by covalent, non covalent or genetic means.

17. The composition according to claim 16, wherein a nucleic acid encoding said peptide is genetically inserted in addition to or in place of one or more residue(s) of a native viral sequence that encodes a polypeptide exposed at the viral surface, so that said peptide is expressed at the surface of the viral particle.

18. The composition according to claim 17, wherein said polypeptide exposed at the viral surface is an adenoviral capsid protein.

19. The composition according to claim 18, wherein said adenoviral capsid protein is selected from the group consisting of fiber, hexon, penton-base and pIX proteins.

20. The composition according to claim 19, wherein said adenoviral capsid protein is a fiber protein and said peptide is genetically inserted into the HI loop or at the C-terminus of said fiber protein.

21. The composition according to claim 19, wherein said fiber protein is further modified.

22. The composition according to claim 21, wherein said modified fiber contains one or more mutation(s) that reduces or abolishes the interaction of said fiber with at least one cellular receptor which normally facilitates virus binding to a cell.

23. The composition according to claim 22, wherein said modified fiber contains one or more mutation(s) that reduces or abolishes the interaction of said fiber with at least the coxsackievirus and adenovirus receptor (CAR).

24. The composition according to claim 23, wherein said modified fiber is an Ad5 fiber comprising the substitution of the serine residue in position 408 by a glutamic acid.

25. The composition according to claim 19, wherein said adenoviral capsid protein is a pIX protein and said peptide is genetically inserted at the C-terminus or within the C-terminal portion of said pIX protein.

26. The composition according to claim 19, wherein said pIX protein is further modified.

27. The composition according to claim 26, wherein said modified pIX is mutated in its coil-coiled domain.

28. The composition according to claim 11, wherein said peptide has the sequence X₁HEWSYLAPYPWFX₂ (SEQ ID NO: 74), wherein each X₁ and X₂ independently of one another represents any amino acid sequence of n amino acids, n varying from 0 to 50 and n being identical or different in X, and X₂.

29. An adenoviral vector comprising a peptide according to any of claim 1, wherein said peptide is exposed at the surface of the viral particle.

30. The adenoviral vector according to claim 29, wherein said peptide is genetically inserted in addition or in place of one or more residue(s) of a native capsid adenoviral protein.

31. The adenoviral vector according to claim 30, wherein said capsid adenoviral protein is selected from the group consisting of fiber, hexon, penton-base and pIX proteins.

32. The adenoviral vector according to claim 31, wherein said adenoviral capsid protein is a fiber protein and said peptide is genetically inserted into the HI loop or at the C-terminus of said fiber protein.

33. The adenoviral vector according to claim 31, wherein said fiber protein is further modified.

34. The adenoviral vector according to claim 33, wherein said modified fiber contains one or more mutation(s) that reduces or abolishes the interaction of said fiber with at least one cellular receptor which normally facilitates virus binding to a cell.

35. The adenoviral vector according to claim 34, wherein said modified fiber contains one or more mutation(s) that reduces or abolishes the interaction of said fiber with at least the coxsackievirus and adenovirus receptor (CAR).

36. The adenoviral vector according to claim 35, wherein said modified fiber is an Ad5 fiber comprising the substitution of the serine residue in position 408 by a glutamic acid.

37. The adenoviral vector according to claim 31, wherein said adenoviral capsid protein is a pIX protein and said peptide is genetically inserted at the C-terminus or within the C-terminal portion of said pIX protein.

38. The adenoviral vector according to claim 31, wherein said pIX protein is further modified.

39. The adenoviral vector according to claim 38, wherein said modified pIX is mutated in its coil-coiled domain.

40. The adenoviral vector according to claim 29, wherein said peptide has the sequence X₁HEWSYLAPYPWFX₂ (SEQ ID NO: 74), wherein each X₁ and X₂ independently of one another represents any amino acid sequence of n amino acids, n varying from 0 to 50 and n being identical or different in X₁ and X₂.

41. The adenoviral vector according to claim 40, wherein said adenoviral vector is an Ad5 adenoviral vector, having the peptide HEWSYLAPYPWF genetically inserted within the HI loop of the adenoviral fiber protein, and wherein said fiber comprises the substitution of the serine residue in position 408 by a glutamic acid.

42. The adenoviral vector according to claim 41, wherein peptide HEWSYLAPYPWF is inserted between residues 545 and 546 of said adenoviral fiber protein.

43. The adenoviral vector according to claim 29, wherein said adenoviral vector is replication-defective.

44. The adenoviral vector according to claim 29, wherein said adenoviral vector is recombinant.

45. A drug for gene transfer, comprising the composition as defined by claim 11.

46. A drug for gene transfer, comprising the adenoviral vector as defined by claim 29.

47. A method for targeting a tumor cell, comprising targeting such cell with an adenoviral vector as defined by claim 29.

48. The method according to claim 47, wherein said tumor cell is a colon tumor cell or a breast tumor cell.

49. A method for the treatment or prevention of a cancer or tumor, comprising administering a therapeutically effective amount of an adenoviral vector according to claim 29 to a patient in need of such treatment.

50. The method according to claim 49, wherein said cancer or tumor is a breast or a colon cancer or tumor.


SEQUENCE LISTING (85 sequences)

SEQ ID NO: 1 - protein, 9 aa, Artificial Sequence
  phage display library
XTHPRFATX

SEQ ID NO: 2 - protein, 9 aa, Artificial Sequence
  phage display library
XRTPFATYX

SEQ ID NO: 3 - protein, 14 aa, Artificial Sequence
  phage display library
XFHVNPTSPTHPLX

SEQ ID NO: 4 - protein, 14 aa, Artificial Sequence
  phage display library
XQTSSPTPLSHTQX

SEQ ID NO: 5 - protein, 9 aa, Artificial Sequence
  phage display library
XPQTSTLLX

SEQ ID NO: 6 - protein, 14 aa, Artificial Sequence
  phage display library
XHLPTSSLFDTTHX

SEQ ID NO: 7 - protein, 9 aa, Artificial Sequence
  phage display library
XVHHLPRTX

SEQ ID NO: 8 - protein, 9 aa, Artificial Sequence
  phage display library
XQLHNHLPX

SEQ ID NO: 9 - protein, 14 aa, Artificial Sequence
  phage display library
XHSFDHLPAAALHX

SEQ ID NO: 10 - protein, 14 aa, Artificial Sequence
  phage display library
XYPSAPPQWLTNTX

SEQ ID NO: 11 - protein, 14 aa, Artificial Sequence
  phage display library
XYPSQSQRXLSXHX

SEQ ID NO: 12 - protein, 9 aa, Artificial Sequence
  phage display library
XTYPSSTLX

SEQ ID NO: 13 - protein, 14 aa, Artificial Sequence
  phage display library
XNTLQVRGVYPSVX

SEQ ID NO: 14 - protein, 14 aa, Artificial Sequence
  phage display library
XYSNRTNTNSHWAX

SEQ ID NO: 15 - protein, 9 aa, Artificial Sequence
  phage display library
XPATNTSKX

SEQ ID NO: 16 - protein, 9 aa, Artificial Sequence
  phage display library
XHVNKLHGX

SEQ ID NO: 17 - protein, 14 aa, Artificial Sequence
  phage display library
XFHVNPTSPTHPLX

SEQ ID NO: 18 - protein, 14 aa, Artificial Sequence
  phage display library
XNANKLWTWVSSPX

SEQ ID NO: 19 - protein, 9 aa, Artificial Sequence
  phage display library
XSGRIPYLX

SEQ ID NO: 20 - protein, 14 aa, Artificial Sequence
  phage display library
XNEDINDVSGRLSX

SEQ ID NO: 21 - protein, 14 aa, Artificial Sequence
  phage display library
XLSPQRASQRLYSX

SEQ ID NO: 22 - protein, 9 aa, Artificial Sequence
  phage display library
XSFSTSPQX

SEQ ID NO: 23 - protein, 9 aa, Artificial Sequence
  phage display library
XERMDSPQX

SEQ ID NO: 24 - protein, 14 aa, Artificial Sequence
  phage display library
XHHGHSPTSPQVRX

SEQ ID NO: 25 - protein, 14 aa, Artificial Sequence
  phage display library
XGSSTGPQRLHVPX

SEQ ID NO: 26 - protein, 14 aa, Artificial Sequence
  phage display library
XTCSLCNPVQPQRX

SEQ ID NO: 27 - protein, 9 aa, Artificial Sequence
  phage display library
XQRLTTLYX

SEQ ID NO: 28 - protein, 14 aa, Artificial Sequence
  phage display library
XWSPGQQRLHNSTX

SEQ ID NO: 29 - protein, 14 aa, Artificial Sequence
  phage display library
XWKSELPVQRARFX

SEQ ID NO: 30 - protein, 14 aa, Artificial Sequence
  phage display library
XSELPSMRLYTQPX

SEQ ID NO: 31 - protein, 14 aa, Artificial Sequence
  phage display library
XHSLHVHKGLSELX

SEQ ID NO: 32 - protein, 14 aa, Artificial Sequence
  phage display library
XSDLPVQLEPERQX

SEQ ID NO: 33 - protein, 14 aa, Artificial Sequence
  phage display library
XTRYLPVLPSLFPX

SEQ ID NO: 34 - protein, 14 aa, Artificial Sequence
  phage display library
XTCSLCNPVQPQRX

SEQ ID NO: 35 - protein, 14 aa, Artificial Sequence
  phage display library
XWEPPVQSAWQLSX

SEQ ID NO: 36 - protein, 14 aa, Artificial Sequence
  phage display library
XHFTFPQQQPPRPX

SEQ ID NO: 37 - protein, 14 aa, Artificial Sequence
  phage display library
XGSTSRPQPPSTVX

SEQ ID NO: 38 - protein, 14 aa, Artificial Sequence
  phage display library
XNFSQPPSKHTRSX

SEQ ID NO: 39 - protein, 14 aa, Artificial Sequence
  phage display library
XQYPHKYTLQPPKX

SEQ ID NO: 40 - protein, 14 aa, Artificial Sequence
  phage display library
XFNQPPSWRVSNSX

SEQ ID NO: 41 - protein, 14 aa, Artificial Sequence
  phage display library
XSVSVGMKPSPRPX

SEQ ID NO: 42 - protein, 14 aa, Artificial Sequence
  phage display library
XSTPRPPLGIPAQX

SEQ ID NO: 43 - protein, 14 aa, Artificial Sequence
  phage display library
XTQSPLNYRPALLX

SEQ ID NO: 44 - protein, 14 aa, Artificial Sequence
  phage display library
XAQSPTIKLTPSWX

SEQ ID NO: 45 - protein, 9 aa, Artificial Sequence
  phage display library
XHNLLTQSX

SEQ ID NO: 46 - protein, 9 aa, Artificial Sequence
  phage display library
XTLVQSPMX

SEQ ID NO: 47 - protein, 14 aa, Artificial Sequence
  phage display library
XNLNTDNYRQLRHX

SEQ ID NO: 48 - protein, 14 aa, Artificial Sequence
  phage display library
XFRPAVHNMPSLQX

SEQ ID NO: 49 - protein, 14 aa, Artificial Sequence
  phage display library
XISRPAPISVDMKX

SEQ ID NO: 50 - protein, 14 aa, Artificial Sequence
  phage display library
XTHRPSLPDSSRAX

SEQ ID NO: 51 - protein, 14 aa, Artificial Sequence
  phage display library
XALHPLTHRHYATX

SEQ ID NO: 52 - protein, 9 aa, Artificial Sequence
  phage display library
XTHRGPQSX

SEQ ID NO: 53 - protein, 14 aa, Artificial Sequence
  phage display library
XSFHMPSRAVSLSX

SEQ ID NO: 54 - protein, 14 aa, Artificial Sequence
  phage display library
XNQSNFTSRALLYX

SEQ ID NO: 55 - protein, 14 aa, Artificial Sequence
  phage display library
XSFPTHIDHHVSPX

SEQ ID NO: 56 - protein, 9 aa, Artificial Sequence
  phage display library
XLNGDPTHX

SEQ ID NO: 57 - protein, 14 aa, Artificial Sequence
  phage display library
XHMPHHVSNLQLHX

SEQ ID NO: 58 - protein, 14 aa, Artificial Sequence
  phage display library
XLPSVSPVLQVLGX

SEQ ID NO: 59 - protein, 14 aa, Artificial Sequence
  phage display library
XDAQQLYLSNWRSX

SEQ ID NO: 60 - protein, 14 aa, Artificial Sequence
  phage display library
XDSYLSSTLPGQLX

SEQ ID NO: 61 - protein, 14 aa, Artificial Sequence
  phage display library
XSPTPTSHQQLHSX

SEQ ID NO: 62 - protein, 14 aa, Artificial Sequence
  phage display library
XAPPGNWRNYLMPX

SEQ ID NO: 63 - protein, 9 aa, Artificial Sequence
  phage display library
XLSNKMSQX

SEQ ID NO: 64 - protein, 14 aa, Artificial Sequence
  phage display library
XMHNVSDSNDSAIX

SEQ ID NO: 65 - protein, 9 aa, Artificial Sequence
  phage display library
XDNSNDLMX

SEQ ID NO: 66 - protein, 14 aa, Artificial Sequence
  phage display library
XTVMEAPRSAILIX

SEQ ID NO: 67 - protein, 11 aa, Artificial Sequence
  phage display library
XCNDIGWVRCX

SEQ ID NO: 68 - protein, 11 aa, Artificial Sequence
  phage display library
XCWPYPSHFCX

SEQ ID NO: 69 - protein, 14 aa, Artificial Sequence
  phage display library
XMPLPQPSHLPLLX

SEQ ID NO: 70 - protein, 14 aa, Artificial Sequence
  phage display library
XLPQRAFWVPPIVX

SEQ ID NO: 71 - protein, 14 aa, Artificial Sequence
  phage display library
XWPVRPWMPGPVVX

SEQ ID NO: 72 - protein, 14 aa, Artificial Sequence
  phage display library
XWPTSPWLEREPAX

SEQ ID NO: 73 - protein, 14 aa, Artificial Sequence
  phage display library
XWPTSPWSSRDWSX

SEQ ID NO: 74 - protein, 14 aa, Artificial Sequence
  phage display library
XHEWSYLAPYPWFX

SEQ ID NO: 75 - protein, 14 aa, Artificial Sequence
  phage display library
XQIDRWFDAVQWLX

SEQ ID NO: 76 - protein, 11 aa, Artificial Sequence
  phage display library
XCLPSTRWTCX

SEQ ID NO: 77 - protein, 11 aa, Artificial Sequence
  phage display library
XCWPMKSXFCX

SEQ ID NO: 78 - protein, 14 aa, Artificial Sequence
  phage display library
XHWAPSMYDYVSWX

SEQ ID NO: 79 - protein, 14 aa, Artificial Sequence
  phage display library
XGHLIPLRQPSHQX

SEQ ID NO: 80 - DNA, 41 bp, Artificial Sequence
  primer
cgattctttagctgccgggcgccgaggcggaggcggaggcg

SEQ ID NO: 81 - DNA, 41 bp, Artificial Sequence
  primer
catagagtatgcacttggactagtgtctcctgtttcctgtg

SEQ ID NO: 82 - DNA, 52 bp, Artificial Sequence
  primer
cgcccggcagccacgagtggagctacctggccccctacccatggttctaagg

SEQ ID NO: 83 - DNA, 52 bp, Artificial Sequence
  primer
cgccttagaaccatgggtagggggccaggtagctccactcgtggctgccggg

SEQ ID NO: 84 - DNA, 42 bp, Artificial Sequence
  primer
ctagtcacgagtggagctacctggccccctacccatggttca

SEQ ID NO: 85 - DNA, 42 bp, Artificial Sequence
  primer
ctagtgaaccatgggtagggggccaggtagctccactcgtga